(19) United States
(12) Patent Application Publication
(10) Pub. No.: US 2023/0219919 A1
(43) Pub. Date: Jul. 13, 2023

(54) LITHIUM EXTRACTION WITH CROWN ETHERS

(71) Applicant: 6TH Wave Innovations Corp, Baltimore, MD (US)

(72) Inventors: Jonathan P. Gluckman, Arnold, MD (US); Garrett Kraft, Cottonwood Heights, UT (US); Glen Southard, Park City, UT (US)

(21) Appl. No.: 17/415,080

(22) PCT Filed: Dec. 17, 2019
(86) PCT No.: PCT/US2019/066985

§ 371 Date: Jun. 17, 2021

Related U.S. Application Data
(60) Provisional application No. 62/780,686, filed on Dec. 17, 2018.

(51) Int. Cl. C07D 323/00; C07F 9/655; C22B 26/12; C22B 3/26

(52) U.S. Cl. CPC C07D 323/00; C07F 9/65527; C22B 3/304; C22B 26/12

(57) ABSTRACT

The present disclosure provides Molecular Recognition Technology (MRT) for selectively sequestering lithium from natural and synthetic brines, leachates, or other chemical mixtures. Also disclosed herein are MRT extractants, ligands, beads and methods of making and using thereof.

### CROSS-REFERENCE TO RELATED APPLICATIONS

This application is a U.S. National Stage Application under 35 U.S.C. § 371 of International Application No. PCT/US2019/066985, filed on Dec. 17, 2019, which claims the benefit of and priority to U.S. Application No. 62/780,686, filed Dec. 17, 2018, which are hereby incorporated by reference in their entireties.

### BACKGROUND

Just a few decades ago, the world demand for lithium was almost non-existent. Since then the growth in lithium production and demand has rapidly increased, driven by the expanding use of lithium ion batteries in portable electronics and electric cars. Lithium is isolated from two primary sources, ore mining and brine extraction, and one secondary source, recycled electronics. Mined high-grade ores, such as spodumene, use roasting and leaching techniques to extract lithium. The isolation of lithium from brines involves large evaporation ponds that can take over a year to process using evaporation, precipitation, adsorption, and ion exchange techniques. Recovery of lithium from brine sources is further complicated by the presence of other ions with similar chemical properties, such as sodium and magnesium, at much higher concentrations. Recycling rate of electronic waste is less than 1% and uses similar techniques to sequester lithium, such as solvent extraction, ion exchange, and/or precipitation. All three sources require extensive processing that are either energy intensive, time demanding, or consumer participation limited, to obtain lithium in a marketable form.

Host-guest chemistry is used to form materials, such as macrocylic ligands, molecularly imprinted polymers, and molecular ion sieves, with specifically designed cavities to substantially improve specificity for a “target” molecule which would be desirable to remove from a process stream (e.g., in waste treatment applications) or to sequester (e.g., isolate) from a process stream because of its value. Molecular recognition technology (MRT) uses macrocyclic ligands, such as crown ethers, lariat ethers, multi-armed ethers, cryptands, calixarenes, and spherands for the formation of molecular ring structures containing chelating sites, within the rings and potentially on pendent groups attached to the rings, to create a cavity that is selective for specific chemical species based on the size of the ring and the chemical composition of the ring and/or pendent groups. MIPs are polymers designed to be highly selective for a specific target molecule. MIPs are prepared by polymerizing a polymerizable ligand which coordinates or “binds” to the target molecule. The target molecule and the polymerizable ligand are incorporated into a prepolymerization mixture, allowed to form a complex, and then polymerized (typically in the presence of one or more non-ligand monomers and a cross-linking monomer). The target molecule thus acts as a “template” to define a cavity or absorption site within the polymerized matrix which is specific to the target molecule (e.g., has a shape or size corresponding to the target molecule). The target molecule is then removed from the MIP prior to its use as an absorbent. Molecular ion sieves or zeolites are generally inorganic materials that create a specific cavity by intercalating a target atom or molecule into its crystal structure. Once the target atoms/molecules are removed, in part or completely, the cavity left behind has a defined size and number of coordination sites for selectively binding to the target atom/molecule.

One example of an untapped source of lithium are geothermal brines. Geothermal brines have difficult operating conditions and have therefore been limited to generating geothermal electricity. Many geothermal brine reservoirs are located deep beneath the earth’s crust and may be under high pressures and temperatures. When these reservoirs are tapped and processed, the conditions are regulated to prevent the brine from destabilizing. These operating conditions may include elevated temperatures (>95° C.), low pH (5-6), managing dissolved solids (30% TDS), omission of oxidizers, and short processing times (<30 minutes). If these conditions aren’t maintained dissolved solids, generally silicates, begin to precipitate out and causes major problems for the processing plant. It is because of the high temperature, low pH, and continuous formation of precipitants that conventional ion-exchange, solvent extraction, and solid phase filtration (e.g. membranes, adsorbent columns) are incompatible with processing the brine. To address these concerns we have developed a composition of matter that may be utilized in the form of solid adsorbents or as extractants for liquid/liquid processing techniques to sequester lithium from lithium containing solutions and is compatible with a variety of harsh conditions, such as those described above for the geothermal brine.

### SUMMARY

The present disclosure relates generally to extractants (e.g., small molecules or polymeric crown ethers) for use in liquid-liquid extraction systems and the functionalization and chemical incorporation of those extractants into solid sorbents for the sequestration of lithium. As such, the present disclosure involves the fields of chemistry, polymers, and materials science.

In one aspect, the present disclosure provides a compound of Formula (I):

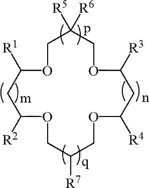

wherein:
R¹, R², R³, and R⁴ are each independently H, alkyl, alkenyl, alkynyl, cycloalkyl, aryl, or heteroaryl, each of which are optionally substituted; or
R¹ and R² and/or R³ and R⁴ taken together with the carbon atoms to which they are attached form a cycloalkyl or aryl ring, each of which is optionally substituted;
R⁵ when present is H, alkyl, alkenyl, alkynyl, or cycloalkyl;
R⁶ when present is —(CH₂)ᵣOH, -(CH₂)ᵣO-alkyl, —OH, -O-alkyl, -O-alkenyl, -O-alkynyl, -O-cycloalkyl; -O-aryl, —O—(CH₂)ₜC(O)OR⁸, —O—(CH₂)ₜS(O)₂OR⁸, —O—(CH₂)ₜS(O)₂N(R⁸)₂, —O—(CH₂)ₜP(O)(OR⁸)₂, —O—(CH₂)ₜC(O)N(R⁹)₂, each of which is optionally substituted;
R⁷ is H, —OH, -O-alkyl, -O-alkenyl, -O-alkynyl, -O-cycloalkyl, —O—(CH₂)ₜC(O)OR⁸, —O—(CH₂)ₜS(O)₂OR⁸, —O—(CH₂)ₜS(O)₂N(R⁸)₂, —O—(CH₂)ₜP(O)(OR⁸)₂, or —O—(CH₂)ₜC(O)N(R⁹)₂;
R⁸ is each independently H, alkyl, haloalkyl, alkenyl, alkynyl, cycloalkyl, aryl, alkylene-cycloalkyl, or alkylene-aryl;
R⁹ is each independently H, alkyl, alkenyl, alkynyl, cycloalkyl, aryl, alkylene-cycloalkyl, alkylene-aryl, or SO₂R¹⁰;
R¹⁰ is alkyl, cycloalkyl, or haloalkyl;
m, n, p, and q are each independently 0 or 1;
r is 1, 2, or 3; and
t is independently 0, 1, or 2;
with the proviso that when p is 0, at least two of R¹, R², R³, and R⁴ are not H.

In one aspect, the present disclosure provides a method of extracting lithium, comprising: (a) mixing a lithium-containing aqueous phase (e.g., a geothermal brine) with an organic phase comprising a suitable organic solvent and one or more of the compounds disclosed herein (e.g., Formula (I), Formula (I-A), Formula (I-B1), Formula (I-B2), Formula (I-C1), Formula (I-C2), Formula (I-C3), Formula (I-D1) and Formula (I-D2)); (b) separating the organic phase and the aqueous phase; and (c) treating the organic phase with aqueous acidic solution to yield a aqueous lithium salt solution.

Thus, in one aspect, the synthesis of MRT based extractants with selectivity towards lithium and their use in solvent extraction systems consisting of an organic phase and an aqueous source phase containing lithium is described herein.

More particularly, the present disclosure relates to an organic phase that may consist of an organic solvent and have dissolved chemical species or suspended particles that promotes the selective transport of lithium from an aqueous source phase to the organic phase.

More particularly, the aqueous phase may be an acidic, basic, or neutral pH and may be in the form of a solution, slurry, or pulp of which may contain one or more types of dissolved ions, suspended particles, precipitates, gange, sediment, or solids.

In one aspect, the present disclosure describes the functionalization of the extractants described herein (e.g., Formula (I), Formula (I-A), Formula (I-B1), Formula (I-B2), Formula (I-C1), Formula (I-C2), Formula (I-C3), Formula (I-D1) and Formula (I-D2) with a polymerizable functionality and incorporation of those extractants into soluble oligomeric molecules for use in solvent extraction systems consisting of an organic phase and an aqueous source phase containing lithium.

In one aspect, the present disclosure provides a polymer of Formula (III), prepared by a process comprising polymerizing a compound of Formula (I-C3) and a compound of Formula (II):

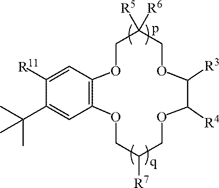

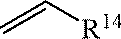

wherein:
R³ and R⁴ are each independently H, alkyl, alkene, optionally substituted aryl or optionally substituted cycloalkyl; or
R³ and R⁴ taken together with the carbon atoms to which they are attached form a cycloalkyl or aryl ring, each of which is optionally substituted;
R⁵ is H or alkyl;
R⁶ is —(CH₂)ᵣOH, -(CH₂)ᵣO-alkyl, —OH, —O—(CH₂)ₜC(O)OR⁸, —O—(CH₂)ₜS(O)₂OR⁸, —O—(CH₂)ₜS(O)₂N(R⁸)₂, —O—(CH₂)ₜP(O)₂(OR⁸)₂, —O—(CH₂)ₜC(O)N(R⁹)₂, each of which is optionally substituted;
R⁷ is H, —OH, -O-alkyl, -O-alkenyl, -O-alkynyl, -O-cycloalkyl, or -O-alkylene-SiR¹³;
R⁸ is each independently H, alkyl, alkenyl, alkynyl, cycloalkyl, aryl, alkylene-cycloalkyl, or alkylene-aryl;
R⁹ is each independently H, alkyl, alkenyl, alkynyl, cycloalkyl, aryl, alkylene-cycloalkyl, alkylene-aryl, or SO₂R¹⁰;
R¹⁰ is alkyl, cycloalkyl, or haloalkyl;
R¹¹ is each independently H, alkyl, haloalkyl, alkene, alkyne, cycloalkyl, or aryl;
R¹³ is H, Cl, OH, alkyl, -O-alkyl, or aryl;
r is 1, 2, or 3;
t is independently 0, 1, or 2;
u is independently 1, 2, or 3;
with the proviso that either R⁷ is -O-alkenyl or -O-alkylene-SiR¹³ or R¹¹ is -alkenyl; and
R¹⁴ is optionally substituted aryl or optionally substituted heteroaryl.

In one aspect, the polymerizable extractants are incorporated into a suspension polymerization to form solid sorbent macroreticular beads with high surface area, high capacity, and high selectivity for lithium. In some embodiments, these solid sorbents are exposed to an aqueous source phase containing lithium for removal and concentration.

More particularly, the solid sorbents refer to incorporation of the extractant into the polymer matrix during the polymerization reaction or as a surface functionalization reaction of organic or inorganic particles, and the as formed solid sorbents are utilized in a batch type or continuous flow column setup.

In one aspect, the present disclosure provides a method of extracting lithium, comprising: (a) mixing a lithium-containing aqueous phase with an organic phase comprising a suitable organic solvent and one or more polymers of Formula (III), the macroreticular beads disclosed herein, a sorbent disclosed herein, or a mixture thereof; (b) separating the organic phase and the aqueous phase; and (c) treating the organic phase with acidic solution to yield a lithium salt solution.

More particularly, the extractants and corresponding MRT technology uses ion exchange principals and as such allows the exchange of lithium with a hydrogen or hydronium ion during elution to form a concentrated lithium solution in all of the systems described when exposed to an acid of sufficient strength for a sufficient period of time.

### BRIEF DESCRIPTION OF THE DRAWINGS

FIG. 1 shows crown-4 macrocyclic ligands, where the electronegative chelating atoms A can be O, S, N-R, or P-R.

FIG. 2 shows chemical structures of various non-limiting embodiments of hydrophobicity adjusted macrocycles.

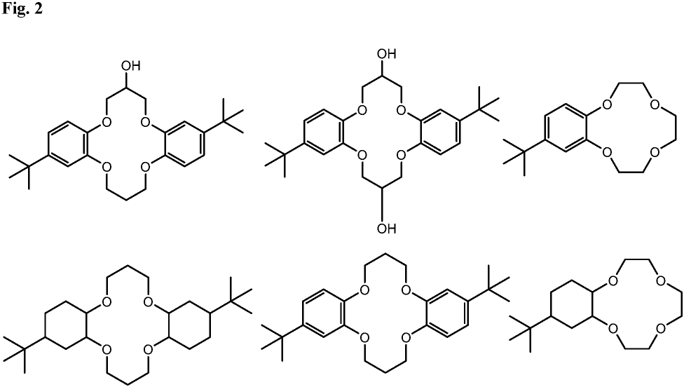

FIG. 3 shows chemical structures of various non-limiting embodiments of single and multi-armed macrocycles with adjusted number of coordination sites.

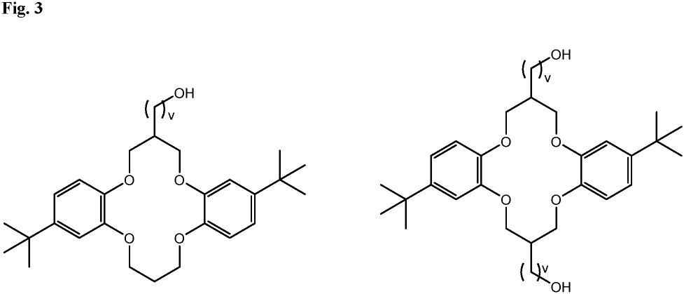

FIG. 4 shows chemical structures of various non-limiting embodiments of macrocycles functionalized with proton-ionizable groups.

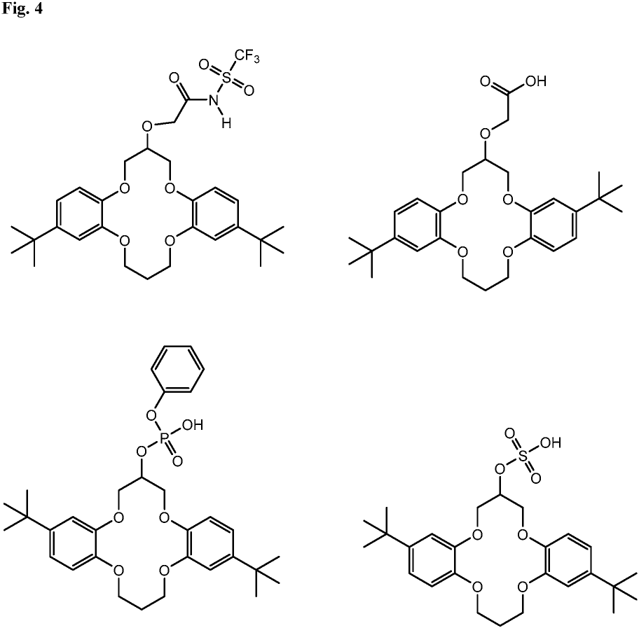

FIG. 5 shows exemplary chemical structures of various non-limiting embodiments of the macrocyclic ligand using multiple design elements such as number of coordination sites, hydrophobicity, proton-ionizable groups, ring size, and composition of electronegative atoms in the ring.

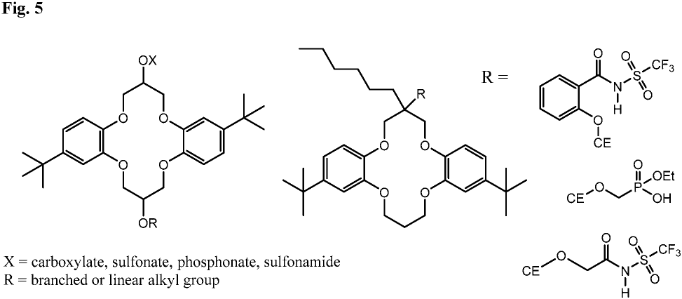

FIG. 6 shows a non-limiting example of an oligomeric extractant combining a monomeric extractant with a vinyl functional group.

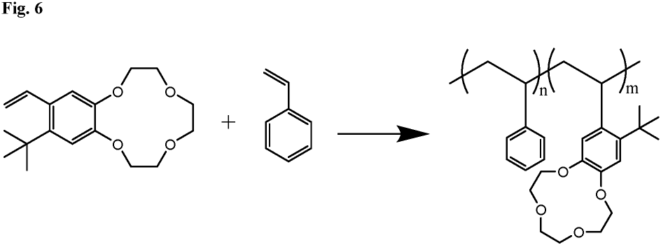

FIG. 7 shows non-limiting examples of polymerizable vinyl and silane functional groups with spacers. X= H, Cl, OH, alkyl, alkoxy, or aromatic.

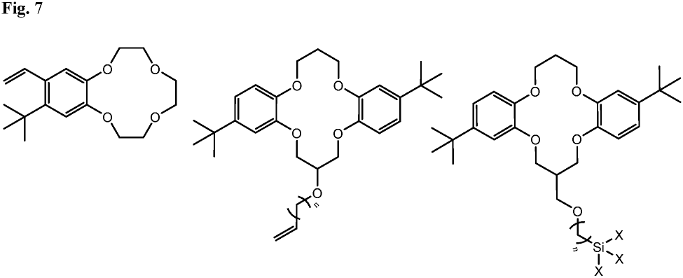

FIG. 8 shows a flow chart describing a representative batch liquid-liquid extraction process of the present disclosure.

FIG. 9 shows a flow chart describing a representative continuous liquid-liquid extraction process of the present disclosure.

FIG. 10 provides graphs of lithium extraction performance of various functional groups in different diluents: (A) monosulfate 3, (B) monocarboxylate 4, (C) disulfonate 11, (D) dicarboxylate 9, and (E) diphosphonate 12. pH was monitored for each extraction.

FIG. 11 shows a graph of the lithium ion selectivity coefficient for various metals during a liquid-liquid extraction of Salton Sea brine with compounds of the present disclosure containing various other metal ions.

FIG. 12 shows a graph comparing the concentration of metal ions in the loaded and stripped organic phase obtained from extraction of Salton Sea brine with Compound 8 in 2-ethylhexanol.

FIG. 13 shows a graph of the lithium ion selectivity coefficient for various metals during a liquid-liquid extraction of Synthetic Chile brine with compounds of the present disclosure.

FIG. 14 provides a graph showing the effects of buffer on maintaining pH during extraction of brine solutions.

FIG. 15 shows an example laboratory-scale apparatus for use in a continuous liquid-liquid extraction of the present disclosure.

### DEFINITIONS

While the following terms are believed to be well understood by one of ordinary skill in the art, the following definitions are set forth to facilitate explanation of the presently disclosed subject matter.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, and the like. The alkylene chain is attached to the rest of the molecule through a single bond and to a radical group (e.g., those described herein) through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), isopropenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to an unsaturated, straight or branched divalent hydrocarbon chain radical having one or more olefins and from two to twelve carbon atoms. Non-limiting examples of C₂-C₁₂ alkenylene include ethenylene, propenylene, n-butenylene, and the like. The alkenylene chain is attached to the rest of the molecule through a single bond and to a radical group (e.g., those described herein) through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to an unsaturated, straight or branched divalent hydrocarbon chain radical having one or more alkynes and from two to twelve carbon atoms. Non-limiting examples of C₂-C₁₂ alkynylene include ethynylene, propynylene, n-butynylene, and the like. The alkynylene chain is attached to the rest of the molecule through a single bond and to a radical group (e.g., those described herein) through a single bond. The points of attachment of the alkynylene chain to the rest of the molecule and to the radical group can be through any two carbons within the chain having a suitable valency. Unless stated otherwise specifically in the specification, an alkynylene chain can be optionally substituted.

“Alkoxy” refers to a group of the formula —ORₐ where Rₐ is an alkyl, alkenyl or alknyl as defined above containing one to twelve carbon atoms. Unless stated otherwise specifically in the specification, an alkoxy group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

“Alkylene-aryl” refers to a radical of the formula —R_{b}—R_{c} where R_{b} is an alkylene, as defined above and R_{c} is one or more aryl radicals as defined above. Examples include benzyl, diphenylmethyl, and the like. Unless stated otherwise specifically in the specification, an aralkyl group can be optionally substituted.

“Carbocyclyl,” “carbocyclic ring” or “carbocycle” refers to a rings structure, wherein the atoms which form the ring are each carbon, and which is attached to the rest of the molecule by a single bond. Carbocyclic rings can comprise from 3 to 20 carbon atoms in the ring. Carbocyclic rings include aryls and cycloalkyl, cycloalkenyl, and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon consisting solely of carbon and hydrogen atoms, which can include fused or bridged ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Alkylene-cycloalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Cycloalkenyl” refers to a stable non-aromatic monocyclic or polycyclic hydrocarbon consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyls include, for example, cyclopentenyl, cyclohexenyl, cycloheptenyl, cycloctenyl, and the like. Polycyclic cycloalkenyls include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl include, for example, cycloheptynyl, cyclooctynyl, and the like. Unless otherwise stated specifically in the specification, a cycloalkynyl group can be optionally substituted.

“Haloalkyl” refers to an alkyl, as defined above, that is substituted by one or more halo radicals, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Heterocyclyl,” “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclycl or heterocyclic rings include heteroaryls, heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused or bridged ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heteroaryl” refers to a 5- to 20-membered ring system comprising hydrogen atoms, one to nineteen carbon atoms, one to six heteroatoms selected from the group consisting of nitrogen, oxygen and sulfur, at least one aromatic ring, and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“Heterocyclylalkyl” refers to a radical of the formula —R_{b}—R_{c} where R_{b} is an alkylene, alkenylene, or alkynylene group as defined above and Rₑ is a heterocyclyl radical as defined above. Unless stated otherwise specifically in the specification, a heterocycloalkylalkyl group can be optionally substituted.

The term “substituted” used herein means any of the groups described herein (e.g., alkyl, alkenyl, alkynyl, alkoxy, aryl, aralkyl, carbocyclyl, cycloalkyl, cycloalkenyl, cycloalkynyl, haloalkyl, heterocyclyl, and/or heteroaryl) wherein at least one hydrogen atom is replaced by a bond to a non-hydrogen atom such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

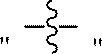

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

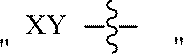

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

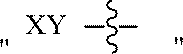

infers that when R³ is “XY”, the point of attachment bond is the same bond as the bond by which R³ is depicted as being bonded to CH₃.

### DETAILED DESCRIPTION OF THE INVENTION

The present disclosure is directed, in various embodiments, to improved methods for preparing Molecular Recognition Technology (MRT) based materials (extractants, sorbents, or other MRT contain materials) MRT materials prepared by such processes, and improved processes utilizing the MRT materials of the present disclosure.

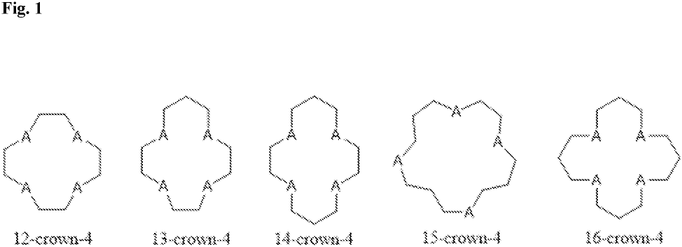

Sorption-based processes are often designed to separate, extract, or sequester a specific molecular species or “target” molecule from a mixture, either to isolate the target molecule (e.g., because of its value), remove a specific specie from a mixture (e.g., because of its toxicity or other hazardous properties), or to detect the target molecule (or molecules associated with the target molecule). Molecular Recognition Technology forms highly selective materials with binding sites specifically tailored to bind to a particular target molecule. Several strategies are used to tailor the MRT materials for the specific target molecule. Innate to all MRT materials is the use of macrocyclic rings to form the ligand or chelating species. The size of the macrocyclic ring is designed to be an ideal fit for the target molecule. A ring that is either too small or too large will result in poor interactions with the ligand and a diminished binding constant (i.e. reduced binding strength). For example, with lithium the 14-crown-4 geometry provides a cavity that is optimized for lithium’s ionic radius. Another aspect of macrocyclic rings is their heterogeneous chemical compositions. In most cases, the ring consists of a carbon based chain with electronegative atoms dispersed throughout. These electronegative atoms generally consist of one or more of O, N, S, and P (FIG. 1). The spacing between the electronegative atoms is not limited, but the most common spacer group is ethylene. For example, one of the most common chemical compositions for macrocyclic rings is poly(ethylene oxide). The number of—CH2CH2O— groups is determined by the size of the target molecule and therefore the size of the ring needed to encompass that molecule. The electronegative atoms act as the primary chelation points in the macrocycle. The purpose of the different types of electronegative atoms is to adjust the electronics of the molecule and the number of chelation or coordination sites. The electronics of the ring can be adjusted by adding chelating atoms that prefer hard ions to the ring, like oxygen, or adjusting it with chelating atoms that prefer soft ions, like sulfur. Lithium is considered a hard ion and therefore binds best with oxygen atoms at the chelating sites. These small tweaks in the electronic structure along with optimizing the number of coordination sites is key to designing the selectivity of the molecule. Additional chelating sites can be added by attaching an arm or another ring to the macrocycle. This can improve the binding strength with the increase in coordination sites or act as a counter charge for an ion, by adding an ionizable group, such as a proton-ionizable group like carboxylate. Lithium complexes are more stable when there are 4-6 coordination sites. Binding a lithium ion to a negatively charged ligand can also form a neutral complex that is more compatible with dissolution in organic phases, and several different extraction techniques.

### Small Molecule Extractants

Lithium has a preference of four planar coordination sites, and as such relates to various embodiments of the 12-crown-4, 13-crown-4, 14-crown-4, 15-crown-4, and 16-crown-4 configurations of the base macrocycles. In these embodiments the chelating sites can consist of one or more of the following: O, S, N-R, or P-R. In a preferred embodiment is the 12-crown-4 ether, and a more preferred embodiment is the 14-crown-4 ether.

In some embodiments, the present disclosure provides a compound of Formula (I):

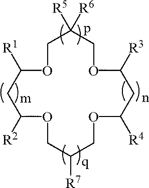

wherein:
R¹, R², R³, and R⁴ are each independently H, alkyl, alkenyl, alkynyl, cycloalkyl, aryl, or heteroaryl, each of which are optionally substituted; or
R¹ and R² and/or R³ and R⁴ taken together with the carbon atoms to which they are attached form a cycloalkyl or aryl ring, each of which is optionally substituted;
R⁵ when present is H, alkyl, alkenyl, alkynyl, or cycloalkyl;
R⁶ when present is —(CH₂)ᵣOH, -(CH₂)ᵣO-alkyl, —OH, -O-alkyl, -O-alkenyl, -O-alkynyl, -O-cycloalkyl; -O-aryl, —O—(CH₂)ₜC(O)OR⁸, —O—(CH₂)ₜS(O)₂OR⁸, —O—(CH₂)ₜS(O)₂N(R⁸)₂, —O—(CH₂)ₜP(O)(OR⁸)₂, —O—(CH₂)ₜC(O)N(R⁹)₂, each of which is optionally substituted;
R⁷ is H, —OH, -O-alkyl, -O-alkenyl, -O-alkynyl, -O-cycloalkyl, —O—(CH₂)ₜC(O)OR⁸, —O—(CH₂)ₜS(O)₂OR⁸, —O—(CH₂)ₜS(O)₂N(R⁸)₂, —O—(CH₂)ₜP(O)(OR⁸)₂, or —O—(CH₂)ₜC(O)N(R⁹)₂;
R⁸ is each independently H, alkyl, haloalkyl, alkenyl, alkynyl, cycloalkyl, aryl, alkylene-cycloalkyl, or alkylene-aryl;
R⁹ is each independently H, alkyl, alkenyl, alkynyl, cycloalkyl, aryl, alkylene-cycloalkyl, alkylene-aryl, or SO₂R¹⁰;
R¹⁰ is alkyl, cycloalkyl, or haloalkyl;
m, n, p, and q are each independently 0 or 1;
r is 1, 2, or 3; and
t is independently 0, 1, or 2;
with the proviso that when p is 0, at least two of R¹, R², R³, and R⁴ are not H.

In some embodiments of Formula (I), when p is 0, at least three of R¹, R², R³, and R⁴ are not H. In some embodiments, when p is 0, none of R¹, R², R³, and R⁴ are H. In some embodiments, when p is 1, at least one of R1, R², R³, and R⁴ is not H. In some embodiments, when p is 1, at least two of R1, R², R³, and R⁴ are not H. In some embodiments, when p is 1, at least three of R1, R², R³, and R⁴ are not H. In some embodiments, when p is 1, none of R¹, R², R³, and R⁴ are H.

In some embodiments of Formula (I), when q is 0, at least two of R¹, R², R³, and R⁴ are not H. In some embodiments, when q is 0, at least three of R¹, R², R³, and R⁴ are not H. In some embodiments, when q is 0, none of R¹, R², R³, and R⁴ are H.

In some embodiments of Formula (I), when p is 0 and q is 0, at least two of R¹, R², R³, and R⁴ are not H. In some embodiments, when p is 0 and q is 0, at least three of R¹, R², R³, and R⁴ are not H. In some embodiments, when p is 0 and q is 0, none of R¹, R², R³, and R⁴ are H. In some embodiments, when p is 1 and q is 0, at least two of R¹, R², R³, and R⁴ are not H. In some embodiments, when p is 0 and q is 1, at least two of R¹, R², R³, and R⁴ are not H. In some embodiments, when p is 1 and q is 0, at least three of R¹, R², R³, and R⁴ are not H. In some embodiments, when p is 0 and q is 1, at least three of R¹, R², R³, and R⁴ are not H.

In some embodiments of Formula (I), m and n are each 0. In some embodiments, m and n are each 1. In some embodiments, m is 1 and n is 0. In some embodiments, m is 0, m is 1.

In some embodiments of Formula (I), p and q are each 1. In some embodiments, p and q are each 0. In some embodiments, p is 1 and q is 0. In some embodiments, p is 0 and q is 1.

In some embodiments of Formula (I), m, n, p, and q are 1. In some embodiments, m, n, p, and q are 0. In some embodiments, m and n are 0 and p and q are 1. In some embodiments, m and n are 1 and p and q are 0. In some embodiments, p is 1 and m, n, and q are 0. In some embodiments, q is 1 and m, n, and p are 0.

In some embodiments of Formula (I), R¹, R², R³ and R⁴ are each independently H, alkyl, alkenyl, optionally substituted aryl or optionally substituted cycloalkyl. In some embodiments, R¹, R², R³ and R⁴ are each independently alkyl, alkenyl, optionally substituted aryl or optionally substituted cycloalkyl. In some embodiments, R¹, R², R³ and R⁴ are each independently optionally substituted aryl or optionally substituted cycloalkyl. In some embodiments, the alkyl is a C₁₋₆alkyl, the alkenyl is a C₂₋₆alkenyl, optionally substituted aryl is optionally substituted phenyl, and the optionally substituted cycloalkyl is optionally substituted cyclohexyl. In some embodiments, R¹ and R² are H. In some embodiments, R³ and R⁴ are H.

In some embodiments of Formula (I), R¹ and R² taken together with the carbon atoms to which they are attached form a cycloalkyl or aryl ring, each of which is optionally substituted. In some embodiments, R¹ and R² taken together with the carbon atoms to which they are attached form an optionally substituted aryl ring. In some embodiments, the cycloalkyl ring is an optionally substituted cyclohexyl. In some embodiments, the aryl ring is an optionally substituted phenyl. In some embodiments, the optional substituent is selected from one or more of the group consisting of halogen, alkyl, haloalkyl, alkenyl, and cycloalkyl. In some embodiments, the halogen is F or Cl; the alkyl is a C₁₋₆alkyl; the haloalkyl is CF₃, CHF₂, CH2F, or CH₂Cl; the alkenyl is a C₂₋₄alkenyl; and the cycloalkyl is a C₃₋₆cycloalkyl. In some embodiments, the C₁₋₆alkyl is methyl, ethyl, propyl, i-propyl, butyl, isobutyl, t-butyl, or t-amyl. In some embodiments, the C₁₋₆alkyl is t-butyl. In some embodiments, the haloalkyl is CH₂Cl. In some embodiments, the C₂₋₄alkenyl is vinyl. In some embodiments, the optionally substituted phenyl is selected from the group consisting of

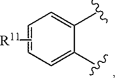

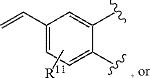

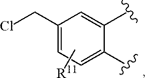

wherein R¹¹ is C₁₋₆alkyl. In some embodiments, the optionally substituted phenyl is selected from the group consisting of

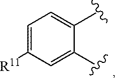

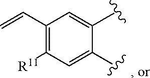

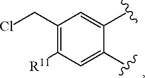

wherein R¹¹ is C₁₋₆alkyl. In some embodiments, the optionally substituted phenyl is selected from the group consisting of

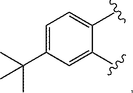

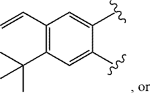

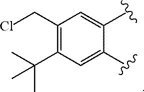

In some embodiments, the optionally substituted cyclohexyl is

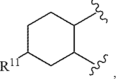

wherein R¹¹ is C₁₋₆alkyl. In some embodiments, the optionally substituted cyclohexyl is

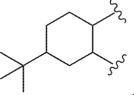

In some embodiments of Formula (I), R³ and R⁴ taken together with the carbon atoms to which they are attached form a cycloalkyl or aryl ring, each of which is optionally substituted. In some embodiments, R³ and R⁴ taken together with the carbon atoms to which they are attached form an aryl ring, each of which is optionally substituted. In some embodiments, the cycloalkyl ring is an optionally substituted cyclohexyl. In some embodiments, the aryl ring is an optionally substituted phenyl. In some embodiments, the optional substituent is selected from one or more of the group consisting of halogen, alkyl, haloalkyl, alkenyl, and cycloalkyl. In some embodiments, the halogen is F or Cl; the alkyl is a C₁₋₆alkyl; the haloalkyl is CF₃, CHF₂, CH2F, or CH₂Cl; the alkenyl is a C₂₋₄alkenyl; and the cycloalkyl is a C₃₋₆cycloalkyl. In some embodiments, the C₁₋₆alkyl is methyl, ethyl, propyl, i-propyl, butyl, isobutyl, t-butyl, or t-amyl. In some embodiments, the C₁₋₆alkyl is t-butyl. In some embodiments, the haloalkyl is CH₂Cl. In some embodiments, the C₂₋₄alkenyl is vinyl. In some embodiments, the optionally substituted phenyl is selected from the group consisting of

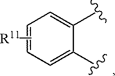

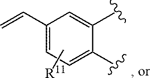

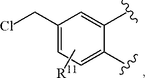

wherein R¹¹ is C₁₋₆alkyl. In some embodiments, the optionally substituted phenyl is selected from the group consisting of

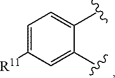

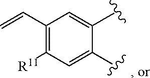

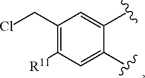

wherein R¹¹ is C₁₋₆alkyl. In some embodiments, the optionally substituted phenyl is selected from the group consisting of

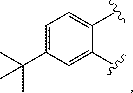

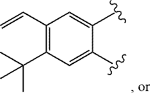

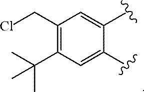

In some embodiments, the optionally substituted cyclohexyl is

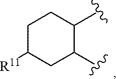

wherein R¹¹ is C₁₋₆alkyl. In some embodiments, the optionally substituted cyclohexyl is

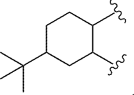

In some embodiments of Formula (I), R⁵ is H or C₁₋₁₀alkyl. In some embodiments, R⁵ is H. In some embodiments, R⁵ is C₁₋₁₀alkyl. In some embodiments, R⁵ is methyl, ethyl, propyl, butyl, pentyl or hexyl. In some embodiments, R⁵ is hexyl. In some embodiments, the R⁵ group is optionally substituted C₁₋₁₀alkyl.

In some embodiments of Formula (I), R⁶ is selected from the group consisting of—(CH₂)ᵣOH, -(CH₂)ᵣO-alkyl, —OS(O)₂OH, —O(CH₂)ₜP(O)(OR⁸)(OH), —O(CH₂)ₜC(O)OH, —O(CH₂)ₜC(O)NH(R⁹) and optionally substituted —OPh. In some embodiments, R⁶ is —(CH₂)ᵣOH, -(CH₂)ᵣO-alkyl. In some embodiments, R⁶ is selected from the group consisting of—OS(O)₂OH, —O(CH₂)ₜP(O)(OR⁸)(OH), —O(CH₂)ₜC(O)OH, —O(CH₂)ₜC(O)NH(R⁹) and optionally substituted —OPh. In some embodiments, R⁶ is —OS(O)₂OH. In some embodiments, R⁶ is —O(CH₂)ₜP(O)(OR⁸)(OH). In some embodiments, R⁶ is —O(CH₂)ₜC(O)OH. In some embodiments, R⁶ is —O(CH₂)ₜC(O)NH(R⁹). In some embodiments, R⁶ is optionally substituted —OPh. In some embodiments, —OPh is optionally substituted with —C(O)N(H)S(O)₂R¹², wherein R¹² is selected from the group consisting of alkyl, haloalkyl, or cycloalkyl. In some embodiments, R¹² is haloalkyl, and the haloalkyl is CF₃. In some embodiments, the optionally substituted phenyl is

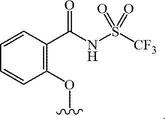

In some embodiments of Formula (I), r is 1 or 2. In some embodiments, r is 1. In some embodiments, r is 2. In some embodiments, r is 3.

In some embodiments of Formula (I), t is 0 or 1. In some embodiments, t is 0. In some embodiments, t is 1. In some embodiments, t is 2.

In some embodiments of Formula (I), R⁷ is H, alkyl, —OH, -O-alkyl, —O—(CH₂)ₜC(O)OR⁸, —O—(CH₂)ₜS(O)₂OR⁸, or —O—(CH₂)ₜP(O)(OR⁸)₂. In some embodiments, R⁷ is H. In some embodiments, R⁷ is alkyl, —OH, or -O-alkyl. In some embodiments, R⁷ is —OH. In some embodiments, R⁷ is —O—alkyl. In some embodiment, the alkyl is C₁₋₁₀alkyl. In some embodiments, the alkyl is hexyl. In some embodiments, R⁷ is —OS(O)₂OH. In some embodiments, R⁷ is —O(CH₂)ₜP(O)(OR⁸)(OH). In some embodiments, R⁷ is —O(CH₂)ₜC(O)OH.

In some embodiments, R⁶ and R⁷ are each —OS(O)₂OH. In some embodiments, R⁶ and R⁷ are each —O(CH₂)ₜP(O)(OR⁸)(OH). In some embodiments, R⁶ and R⁷ are each —O(CH₂)ₜC(O)OH. In some embodiments, R⁶ is —O(CH₂)ₜP(O)(OR⁸)(OH) and R⁷ is H. In some embodiments, R⁶ is —O(CH₂)ₜC(O)OH and R⁷ is H. In some embodiments, R⁶ is —O(CH₂)ₜC(O)NH(R⁹) and R⁷ is H. In some embodiments, R⁶ is optionally substituted —OPh and R⁷ is H. In some embodiments, R⁶ is

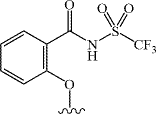

and R⁷ is H. In some embodiments, R⁶ is —O(CH₂)ₜP(O)(OR⁸)(OH) and R⁷ is —OH. In some embodiments, R⁶ is —O(CH₂)ₜC(O)OH and R⁷ is —OH. In some embodiments, R⁶ is —O(CH₂)ₜC(O)NH(R⁹) and R⁷ is —OH. In some embodiments, R⁶ is optionally substituted —OPh and R⁷ is —H. In some embodiments, R⁶ is

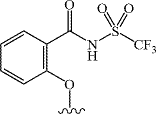

and R⁷ is -H. In some embodiments, R⁶ is —O(CH₂)ₜP(O)(OR⁸)(OH) and R⁷ is -O-C₁₋₁₀alkyl. In some embodiments, R⁶ is —O(CH₂)tC(O)OH and R⁷ is -O-C₁₋₁₀alkyl. In some embodiments, R⁶ is —O(CH₂)tC(O)NH(R⁹) and R⁷ is -O-C₁₋₁₀alkyl. In some embodiments, R⁶ is optionally substituted —OPh and R⁷ is -O-C₁₋₁₀alkyl. In some embodiments, R⁶ is

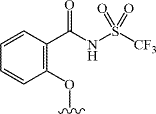

and R⁷ is -O-C₁₋₁₀alkyl. In some embodiments, the alkyl is hexyl. In some embodiments, R⁶ is —(CH₂)ᵣOH and R⁷ is —(CH₂)ᵣOH, wherein r is 0 or 1. In some embodiments, r is 1.

In some embodiments of Formula (I), R⁸ is each independently H, C₁₋₅alkyl or aryl. In some embodiments, C₁₋₅alkyl is methyl, ethyl, isopropyl, or t-butyl. In some embodiments, R⁸ is each independently H, ethyl or phenyl. In some embodiments, R⁸ is each independently H or ethyl. In some embodiments, R⁸ is each independently H or phenyl.

In some embodiments of Formula (I), R⁹ is SO₂R¹⁰, and R¹⁰ is C₁₋₅alkyl or haloalkyl. In some embodiments, R⁹ is SO₂R¹⁰, and R¹⁰ is C₁₋₅alkyl or haloalkyl selected from the group consisting of CF₃, CHF₂, and CH₂F. In some embodiments, R⁹ is SO₂R¹⁰, and R¹⁰ is haloalkyl selected from the group consisting of CF₃, CHF₂, and CH₂F. In some embodiments, R⁹ is SO₂R¹⁰, and R¹⁰ is CF₃.

In some embodiments, the present disclosure provides a compound of Formula (I-A):

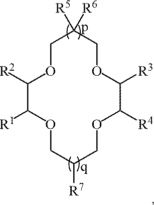

wherein R¹, R², R³, R⁴, R⁵, R⁶, R⁷, p, and q are as defined above for Formula (I).

In some embodiments, the present disclosure provides a compound of Formula (I-B1) or Formula (I-B2):

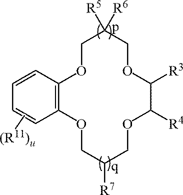

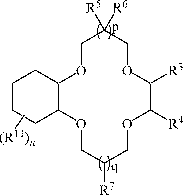

wherein R³, R⁴, R⁵, R⁶, R⁷, p, and q are as defined above for Formula (I).

In some embodiments of Formula (I-B1) and Formula (I-B2), R¹¹ is each independently H, alkyl, haloalkyl, alkenyl, alkynyl, cycloalkyl, or aryl. In some embodiments, each R¹¹ is independently H, alkyl, alkenyl, or haloalkyl. In some embodiments, each R¹¹ is independently alkyl, alkenyl, or haloalkyl. In some embodiments, each R¹¹ is independently alkyl or alkenyl. In some embodiments, each R¹¹ is independently alkyl or haloalkyl. In some embodiments, the alkyl is a C₁₋₆alkyl. In some embodiments, the C₁₋₆alkyl is selected from the group consisting of methyl, ethyl, propyl, isopropyl, isobutyl, t-butyl, or isoamyl. In some embodiments, the C₁₋₆alkyl is t-butyl. In some embodiments, the alkenyl is a C₂₋₆alkenyl. In some embodiments, the C₂₋₆alkenyl is vinyl. In some embodiments, the haloalkyl is CH₂Cl.

In some embodiments of Formula (I-B1) and Formula (I-B2), u is 0, 1, 2, or 3. In some embodiments, u is 1, 2, or 3. In some embodiments, u is 1 or 2. In some embodiments, u is 1. In some embodiments, u is 2.

In some embodiments of Formula (I-B1) and Formula (I-B2), u is 1 and R¹¹ is t-butyl. In some embodiments, u is 2 and R¹¹ is CH₂Cl and t-butyl. In some embodiments, u is 2 and R¹¹ is vinyl and t-butyl. In some embodiments, m, n, p, and q are 0. In some embodiments, m and n are 0 and p and q are 1.

In some embodiments, the compound of Formula (I-B1) is selected from the group consisting of:

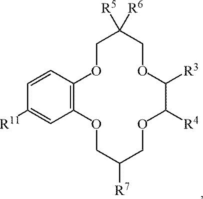

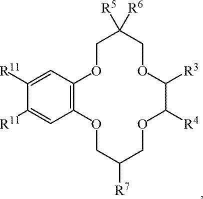

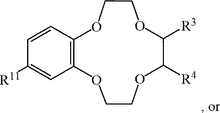

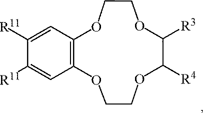

wherein R³, R⁴, R⁵, R⁶, R⁷ are as defined above for Formula (I).

In some embodiments, the compound of Formula (I-B1) is selected from the group consisting of:

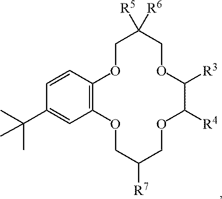

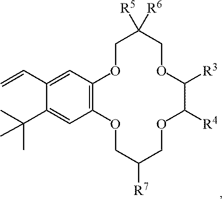

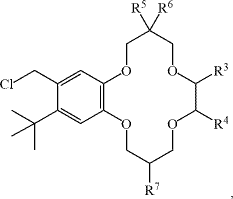

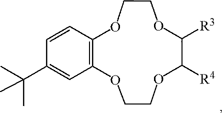

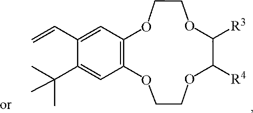

wherein R³, R⁴, R⁵, R⁶, R⁷ are as defined above for Formula (I).

In some embodiments, the present disclosure provides a compound of Formula (I-C1) or Formula (I-C2):

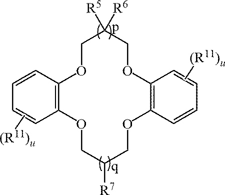

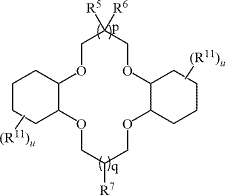

wherein R⁵, R⁶, R⁷, p, and q are as defined above for Formula (I).

In some embodiments of Formula (I-C1) and Formula (I-C2), R¹¹ is each independently H, alkyl, haloalkyl, alkenyl, alkynyl, cycloalkyl, or aryl. In some embodiments, each R¹¹ is independently H, alkyl, alkenyl, or haloalkyl. In some embodiments, each R¹¹ is independently alkyl, alkenyl, or haloalkyl. In some embodiments, each R¹¹ is independently alkyl or alkenyl. In some embodiments, each R¹¹ is independently alkyl or haloalkyl. In some embodiments, the alkyl is a C₁₋₆alkyl. In some embodiments, the C₁₋₆alkyl is selected from the group consisting of methyl, ethyl, propyl, isopropyl, isobutyl, t-butyl, or isoamyl. In some embodiments, the C₁₋₆alkyl is t-butyl. In some embodiments, the alkenyl is a C₂-₆alkenyl. In some embodiments, the C₂-₆alkenyl is vinyl. In some embodiments, the haloalkyl is CH₂Cl.

In some embodiments of Formula (I-C1) and Formula (I-C2), u is 0, 1, 2, or 3. In some embodiments, u is 1, 2, or 3. In some embodiments, u is 1 or 2. In some embodiments, u is 1. In some embodiments, u is 2.

In some embodiments of Formula (I-C1) and Formula (I-C2), u is 1 and R¹¹ is t-butyl. In some embodiments, u is 2 and R¹¹ is CH₂Cl and t-butyl. In some embodiments, u is 2 and R¹¹ is vinyl and t-butyl. In some embodiments, m, n, p, and q are 0. In some embodiments, m and n are 0 and p and q are 1.

In some embodiments, the compound of Formula (I-C1) is selected from the group consisting of:

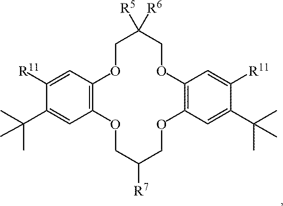

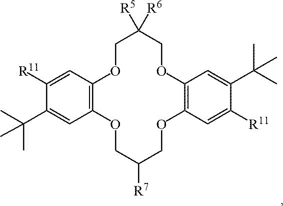

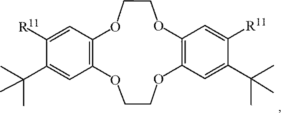

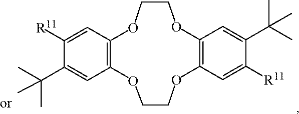

wherein R⁵ and R⁶ are as defined above for Formula (I).

In some embodiments, the present disclosure provides a compound of Formula (I-D1) or Formula (I-D2):

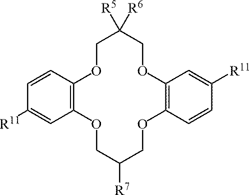

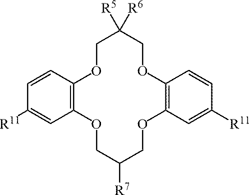

wherein R⁵, R⁶, and R¹¹ are as defined above for Formula (I) and Formula (I-B1).

In some embodiments, present disclosure provides a compound selected from the group consisting of:

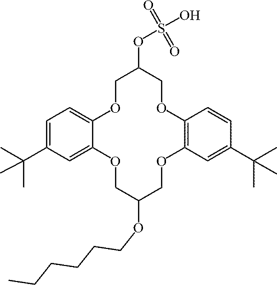

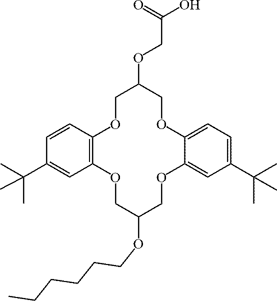

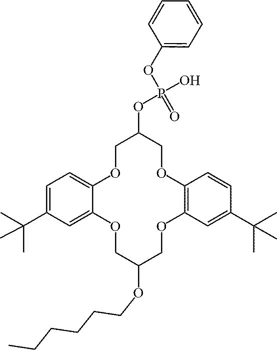

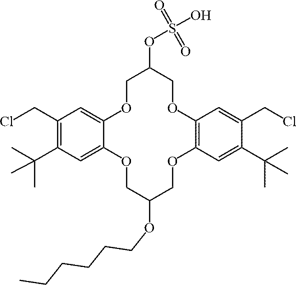

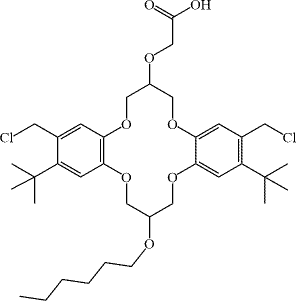

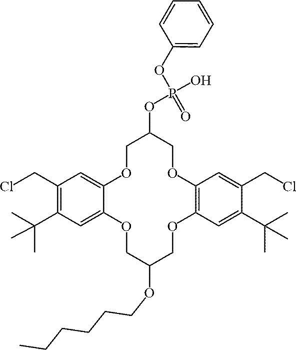

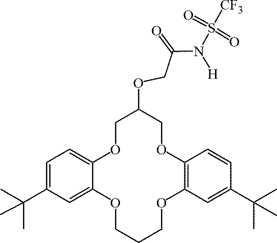

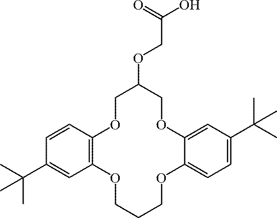

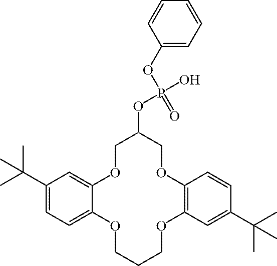

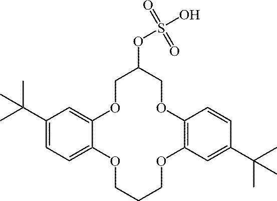

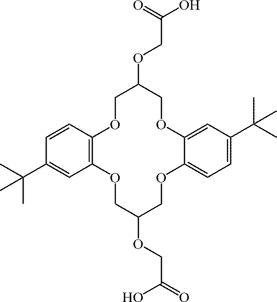

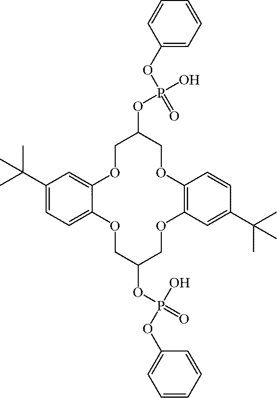

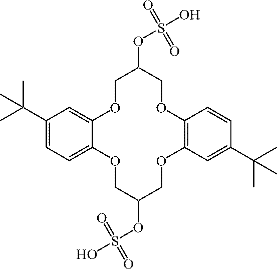

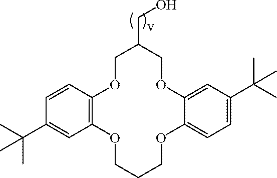

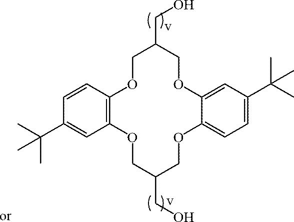

wherein each v is independently 0, 1, 2, or 3.

In some embodiments, present disclosure provides a compound selected from the group consisting of:

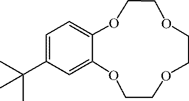 [SMILES: CC(C)(C)c1ccc2c(c1)OCCOCCOCCO2]

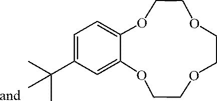

In some embodiments, a compound of Formula (I), Formula (I-A), Formula (I-B1), Formula (I-B2), Formula (I-C1), Formula (I-C2), Formula (I-C3), Formula (I-D1) or Formula (I-D2) has a selectivity coefficient for lithium ion of from 1 to 10, e.g., a selectivity coefficient of 1, a selectivity coefficient of 2, a selectivity coefficient of 3, a selectivity coefficient of 4, a selectivity coefficient of 5, a selectivity coefficient of 6, a selectivity coefficient of 7, a selectivity coefficient of 8, a selectivity coefficient of 9, or a selectivity coefficient of 10. In some embodiments, the compounds of the present disclosure have a selectivity coefficient greater than about 1. In some embodiments, the compounds of the present disclosure have a selectivity coefficient greater than about 3. In some embodiments, the compounds of the present disclosure have a selectivity coefficient greater than about 5. In some embodiments, the compounds of the present disclosure have a selectivity coefficient greater than about 7. In some embodiments, the compounds of the present disclosure have a selectivity coefficient for lithium ion of greater than about 10.

As used throughout the present disclosure, the term “selectivity coefficient” is meant to define a dimensionless value for the ability of a disclosed compound to selectively remove a target ion (e.g., lithium) from an aqueous feed solution (e.g., a geothermal brine) containing one or more other metal ions (e.g., Na, Mg, K, Ca, etc.). It can be used with a number of different measured values (concentration, mass, moles, etc.) to yield the same number. For example, a ratio of lithium (Li) to sodium (Na) in the aqueous acidic solution of 8 means that there is 8X more lithium in the solution by mass, moles, concentration, etc. than sodium. This can be compared to mass ratio in the feed solution to further evaluate the effectiveness of the liquid-liquid extraction method. In some embodiments, the selectivity coefficient is a ratio of lithium to other metal after purification normalized by the lithium/metal ratio in the feed (e.g., geothermal brine). Such a value would be provided by the following equation:

$$\left({\left[\text{Li}\right]}_{\text{purified}} / {\left[\text{metal}\right]}_{\text{purified}}\right) / \left({\left[\text{Li}\right]}_{\text{feed}} / {\left[\text{metal}\right]}_{\text{feed}}\right)$$

As described herein, the hydrophobicity of the macrocycle can be adjusted by adding linear or branched or cyclic alkyl, alkoxy, hydroxyl, ether, polyether, amine, polyamine, benzyl, or aromatic groups attached to one or more atoms in the macrocycle. In a preferred embodiment is 4-hydroxyl-bis(4′-t-butyl)dibenzo-14-crown-4 ether, and 4,11-dihydroxyl-bis(4′-t-butyl)dibenzo-14-crown-4 ether. In a more preferred embodiment is (4′-t-butyl)benzo-12-crown-4 ether, (4′-t-butyl)cyclohexyl-12-crown-4 ether, bis(4′-t-butyl)dibenzo-14-crown-4 ether, or bis(4′-t-butyl)dicyclohexyl-14-crown-4 ether.

In another embodiment, the number of coordination sites of the macrocycle can be adjusted by adding alkyl and aromatic hydroxyl, thiol, amine, polyamine, phosphate, ether, polyether, sulfate, ketone, aldehyde, carbamate, or thiolcarbamate groups attached to one or more atoms in the macrocycle. This can manifest as lariat ethers, multiarmed ethers, cryptands, calixarenes, and spherands. In a preferred embodiment is 4-alkylhydroxyl-bis(4′-t-butyl)dibenzo-14-crown-4 ether, and 4,11-dialkylhydroxyl-bis(4′-t-butyl)dibenzo-14-crown-4 ether.

In another embodiment, a proton-ionizable group can be attached to one or more atoms in the macrocycle to add additional chelating sites and to provide a counter charge for lithium ion, forming a neutral complex. In a preferred embodiment is sym(4′-t-butyl)dibenzo-14-crown-4-oxyacetic acid ether, sym(4′-t-butyl)dibenzo-14-crown-4-oxysulfuric acid ether, sym(4′-t-butyl)dibenzo-14-crown-4-oxyphenylphosphonic acid ether, sym(4′-t-butyl)dibenzo-14-crown-4-oxy-N-((trifluoromethyl)sulfonyl)acetamide ether.

In another embodiment, one or more design elements from the previous embodiments may be used to optimize chemical and physical properties along with performance. Molecule design elements include but are not limited to: ring size, number of chelating sites, type of atom at the chelating sites, proton ionizable groups, functionalities to adjust hydrophobicity, and functional groups capable of undergoing polymerization. In some embodiments, the performance of the small molecule extracts disclosed herein is optimal at a pH of about 9. In some embodiments, the performance of the small molecule extracts disclosed herein is optimal at a pH between about 5.5 to about 7. In some embodiments, the performance of the small molecule extracts disclosed herein is optimal at a pH between about 7 to about 8.

### Solid Sorbents Comprising Small Molecule Extractants

In some embodiments, the present disclosure provides a sorbent comprising a solid support and a compound (small molecule extractant) of Formula (I), Formula (I-A), Formula (I-B1), Formula (I-B2), Formula (I-C1), Formula (I-C2), Formula (I-C3), Formula (I-D1) or Formula (I-D2).

In some embodiments, the compound (i.e., small molecule extractant is selected from the group consisting of:

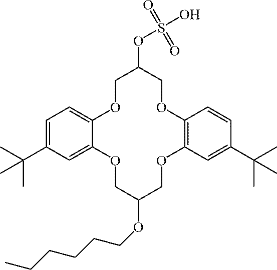

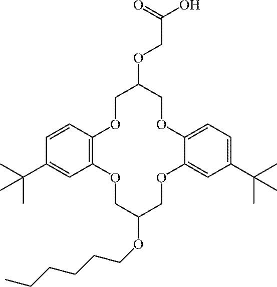

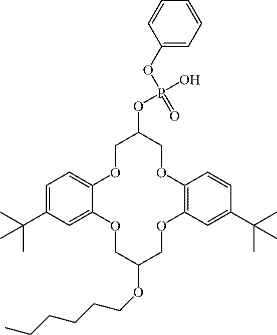

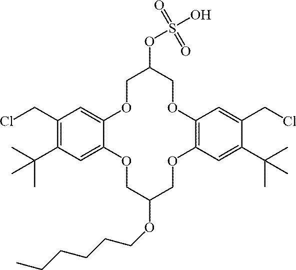

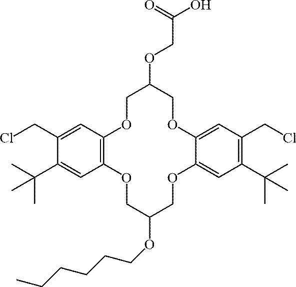

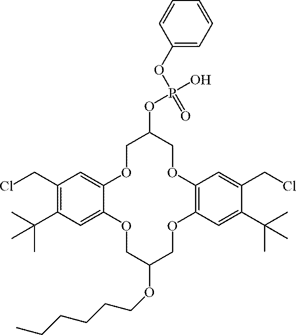

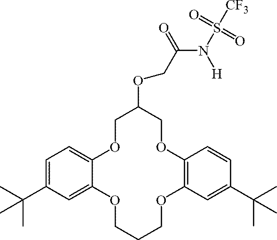

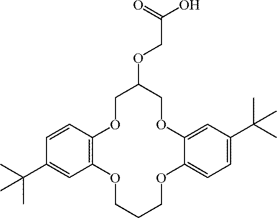

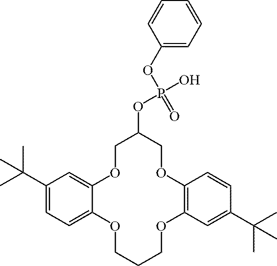

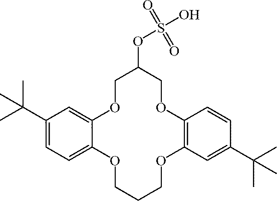

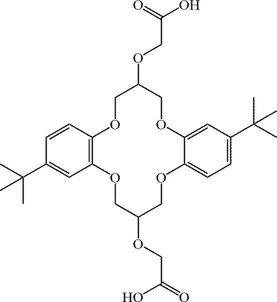

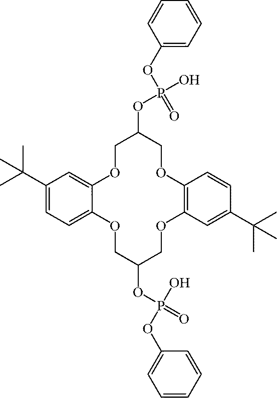

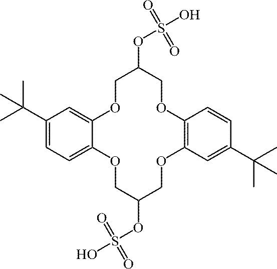

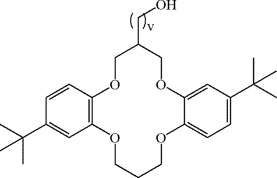

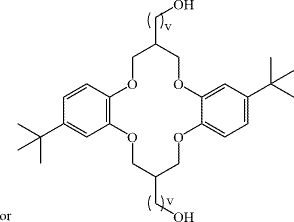

wherein each v is independently 0, 1, 2, or 3.

In some embodiments of the present disclosure, the compound of Formula (I), Formula (I-A), Formula (I-B1), Formula (I-B2), Formula (I-C1), Formula (I-C2), Formula (I-C3), Formula (I-D1) or Formula (I-D2) is coated on a solid support. In some embodiments, the compound is chemically linked to a solid support.

In some embodiments, the solid support is selected from the group consisting of silica, alumina, titania, manganese oxide, glass, zeolite, lithium ion sieve, molecular sieve, or other metal oxide.

In some embodiments, the sorbent has a surface area of about 0.1-500 m²/g. In some embodiments, the sorbent has a surface area of about 0.1-10 m²/g. In some embodiments, the sorbent has a surface area of about 10-100 m²/g. In some embodiments, the sorbent has a surface area of about 100-500 m²/g.

In some embodiments, the sorbent has an average particle size of from about 250 µm to about 5 mm. In some embodiments, the sorbent has an average particle size of from about 250 µm to about 1 mm. In some embodiments, the sorbent has an average particle size of from about 1 mm to about 5 mm. In some embodiments, the sorbent has an average particle size of from about 1 mm to about 3 mm. In some embodiments, the sorbent has an average particle size of from about 3 mm to about 5 mm.

In some embodiments, the use of the sorbent in at least ten lithium ion extraction elution cycles at a temperature of about 100° C. provides less than about 10% compound degradation.

In some embodiments, the use of the sorbent in at least thirty lithium ion extraction elution cycles at a temperature of about 100° C. provides less than about 10% compound degradation.

In some embodiments, the use of the sorbent in at least one hundred lithium ion extraction elution cycles using an extraction temperature of about 100° C. provides less than about 10% compound degradation.

In some embodiments, the use of the sorbent in at least ten lithium ion extraction elution cycles with a source phase having a pH of about 5 to 6 provides less than about 10% compound degradation.

In some embodiments, the use of the sorbent in at least thirty lithium ion extraction elution cycles with a source phase having a pH of about 5 to 6 provides less than about 10% compound degradation.

In some embodiments, the use of the sorbent in at least one hundred lithium ion extraction elution cycles with a source phase having a pH of about 5 to 6 provides less than about 10% compound degradation.

In some embodiments, the flash point of the compound of Formula (I), Formula (I-A), Formula (I-B1), Formula (I-B2), Formula (I-C1), Formula (I-C2), Formula (I-C3), Formula (I-D1) or Formula (I-D2) is > 80° C.

In some embodiments, the selectivity coefficient of the sorbent for the target metal ion greater than about 5. In some embodiments, the selectivity coefficient of the sorbent for the target metal ion greater than about 10. In some embodiments, the target metal ion is lithium.

### Methods of Lithium Sequestration With Small Molecule Extractants

In some embodiments, the present disclosure provides a method of extracting lithium, comprising:
(a) mixing a lithium-containing aqueous phase with an organic phase comprising a suitable organic solvent and one or more compounds of Formula (I), Formula (I-A), Formula (I-B1), Formula (I-B2), Formula (I-C1), Formula (I-C2), Formula (I-C3), Formula (I-D1) or Formula (I-D2);
(b) separating the organic phase and the aqueous phase; and
(c) treating the organic phase with aqueous acidic solution to yield a aqueous lithium salt solution.

In some embodiments, the mixing of step (a) comprises stirring the mixture of the aqueous phase and the organic phase. In some embodiments, the mixing involves contacting the aqueous phase and the organic phase for a period of from about 1 second to about 60 minutes. In some embodiments, the mixing involves contacting the aqueous phase and the organic phase for a period of from about 1 second to about 30 minutes. In some embodiments, the mixing involves contacting the aqueous phase and the organic phase for a period of from about 1 second to about 15 minutes. In some embodiments, the mixing involves contacting the aqueous phase and the organic phase for a period of from about 5 minutes to about 50 minutes. In some embodiments, the mixing involves contacting the aqueous phase and the organic phase for a period of from about 5 minutes to about 15 minutes. In some embodiments, the mixing involves contacting the aqueous phase and the organic phase for a period of from about 10 minutes to about 15 minutes.

In some embodiments of the present method, the suitable organic solvent is selected from the group consisting of alcohols, aldehydes, alkanes, amines, amides, aromatics, carboxylic acids, ethers, ketones, phosphates, or siloxanes or a mixture thereof. In some embodiments, the suitable organic solvent is selected from the group consisting of Exxsol D110™, Orfom SX 11™, and Orfor SX 12™. In some embodiments, the suitable organic solvent is an aromatic solvent (e.g., a heavy aromatic solvent), kerosene, Varsol™ (mixture of aliphatic, open-chain C7-C12 hydrocarbons), octanol, or a mineral oil. In some embodiments, the aromatic solvent has an aromatic content greater than about 40%, greater than about 50%, greater than about 60%, greater than about 70%, greater than about 80%, or greater than about 90%. In some embodiments, the aromatic content is greater than about 99%. In some embodiments, the heavy aromatic solvent is Aromatic 200 (e.g., ExxonMobile Aromatic 200™; Solvesso 200™) or any other heavy aromatic solvent known in the art. Aromatic 200™ solvent is an aromatic hydrocarbon solvent primarily in the range of C12-C15 hydrocarbons. Other non-limiting examples include Aromatic 150 (e.g., ExxonMobile Aromatic 150™; Solvesso 150™) and those that contain C8 hydrocarbons or higher.

In some embodiments of the present method, the organic solvent is 2-ethyl-1-hexanol.

In some embodiments of the present method, the aqueous phase is selected from the group consisting of natural brine, a dissolved salt flat, seawater, concentrated seawater, desalination effluent, a concentrated brine, a processed brine, a geothermal brine, liquid from an ion exchange process, liquid from a solvent extraction process, a synthetic brine, leachate from ores, leachate from minerals, leachate from clays, leachate from recycled products, leachate from recycled materials, or combination thereof. In some embodiments, the aqueous phase is a geothermal brine. In some embodiments, the geothermal brine is Salton Sea brine or Synthetic Chile brine.

In the case of geothermal brines from the Salton Sea the brine is stable at pH levels of less than 7. Thus, in some embodiments, the aqueous phase has an initial pH (or a target operating pH) in the range of about 5.5 to about 7. In other embodiments, the aqueous phase has an initial pH (or target operating pH) in the range of about 5.5 to about 6.5. For other brine sources, such as Synthetic Chile brine, the operating pH is about 7 to 8. Accordingly, in some embodiments, the aqueous phase has an initial pH (or a target operating pH) in the range of about 7 to about 8. In some embodiments, the pH is maintained in these ranges by adding an external acid, base, or buffering agent.

In some embodiments, controlling pH of the aqueous phase is critically important for the disclosed liquid-liquid extraction method. Brine chemical composition and concentration determine the stability and operating pH of the system. Generally speaking, increasing the pH of the brine will lead to increased salt precipitation and destabilization of the brine. Since the extractant materials disclosed herein work on an ion-exchange mechanism, pH is a major factor that contributes to their effectiveness. The extraction process occurs at a higher pH than the elution process, but during elution a proton is exchanged with lithium in the extractant, which is then transported back to the extraction stage where once released, the proton can impact the pH of the brine, decreasing it and potentially reducing the effectiveness of the extraction. Therefore, the pH is monitored during the extraction phase and can be adjusted or controlled with external acid, base, or buffering agent.

In some embodiments of the present method, the aqueous phase further comprises a pH buffer. In some embodiments, the buffer is an acetic acid or a citric acid buffer.

In some embodiments of the present method, the one or more compounds disclosed herein are loaded in a range of from about 1% to about 15% by weight per volume (w/v) of the organic phase, e.g., about 1%, about 2%, about 3%, about 4%, about 5%, about 6%, about 7%, about 8%, about 9%, about 10%, about 11%, about 12%, about 13%, about 14%, or about 15%. In some embodiments, the one or more compounds are loaded in a range of from about 1% to about 5%. In some embodiments, the one or more compounds are loaded in a range of from about 5% to about 10%. In some embodiments, the one or more compounds are loaded in a range of from about 10% to about 15%.

In some embodiments, the temperature of the extraction process is maintained from about 75° C. to about 125° C.

In some embodiments of the present method, the separated organic phase of step (b) comprises a compound disclosed herein and a concentration of lithium ions. In some embodiments, the separated organic layer comprises a compound disclosed herein complexed to a lithium ion.

In some embodiments, the separated organic phase of step (b) is washed with additional (i.e., clean) water.

In some embodiments, treating the separated organic phase of step (b) with an aqueous acidic solution of step (c) involves contacting (e.g., mixing, stirring, agitating, etc.) the organic phase with aqueous acid for a period of from about 1 second to about 60 minutes. In some embodiments, the contacting is for a period of from about 1 second to about 30 minutes. In some embodiments, the contacting is for a period of from about 1 second to about 15 minutes. In some embodiments, the contacting is for a period of from about 5 minutes to about 30 minutes. In some embodiments, the contacting is for a period of from about 5 minutes to about 15 minutes. In some embodiments, the contacting is for a period of from about 10 minutes to about 15 minutes.

In some embodiments, the aqueous acid solution comprises hydrochloric acid, sulfuric acid, phosphoric acid, hydrobromic acid, chloric acid, perchloric acid, nitric acid, formic acid, acetic acid, carbonic acid, or a combination thereof. In some embodiments, the concentration of the aqueous acid solution is from about 0.5 M to about 2 M. In some embodiments, the concentration of the aqueous acid solution is from about 0.5 M to about 1 M. In some embodiments, the concentration of the aqueous acid solution is about 0.5 M. In some embodiments, the concentration of the aqueous acid solution is about 1 M.

Washing the organic phase with aqueous acid, as described in step (c) of the present method, results in liberation of the sequestered lithium from the crown ether. In some embodiments, the present method further comprises treating the organic phase remaining after step (c) with a second volume of aqueous acidic solution to yield a second aqueous lithium salt solution. In some embodiments, the second wash results in enrichment of lithium in the (combined) aqueous acidic solution.

Accordingly, in some embodiments, after washing the organic phase with one or more volumes of aqueous acid solution, the organic phase is recycled for further use. The recycled organic phase that contains a concentration (e.g., 1% to about 15% w/v) of one or more compounds of the present disclosure can be mixed with untreated aqueous feed solution (e.g., geothermal brine) as described in step (a) in order to improve the efficiency and economics of the liquid-liquid extraction method.

In some embodiments of the present method, the aqueous acidic solution comprises about 1% to about 100% (e.g., about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%) of the lithium originally in the metal ion-containing aqueous phase (e.g., geothermal brine). In some embodiments, the aqueous acidic solution comprises about 1% to about 50% of the lithium originally in the metal ion-containing aqueous phase (e.g., geothermal brine). In some embodiments, the aqueous acidic solution comprises about 1% to about 40% of the lithium originally in the metal ion-containing aqueous phase (e.g., geothermal brine). In some embodiments, the aqueous acidic solution comprises about 1% to about 30% of the lithium originally in the metal ion-containing aqueous phase (e.g., geothermal brine).

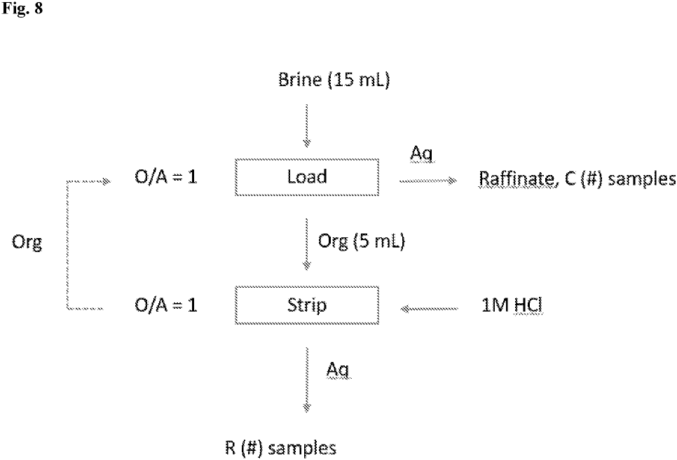

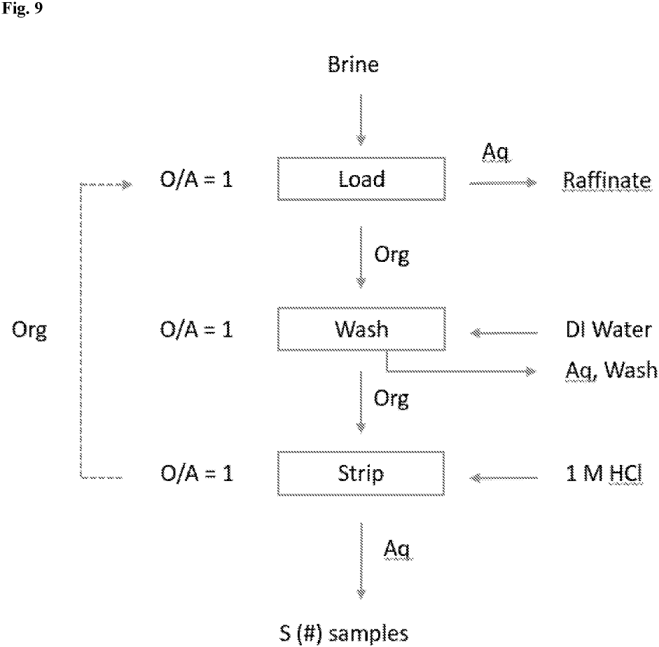

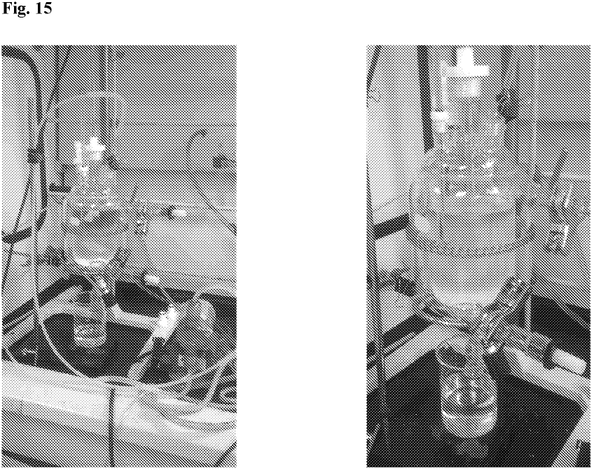

In some embodiments of the present method, the extraction is carried out under batch conditions. In some embodiments, the batch conditions are described by the non-limiting example shown in FIG. 8. In some embodiments, the extraction is carried out under continuous conditions. In some embodiments, the continuous (or continuous flow) conditions are described by the non-limiting example shown in FIG. 9. FIG. 15 provides an example of lab-scale continuous extractor that can be used to carry out the liquid-liquid extraction methods of the present disclosure. As shown in FIGS. 8 and 9, brine feed (e.g., Salton Sea brine or Synthetic Chile brine) is introduced into the system. Organic phase comprising a compound (or polymer, sorbent, etc.) of the present disclosure is mixed with the aqueous phase, which results in a target ion (e.g., lithium) becoming complexed with the chelating moieties of the crown ether. The organic phase comprising targeted metal ion (load) is separated from the raffinate and can be optionally washed with DI water before being stripped with aqueous acid (e.g., 0.5 M or 1.0 M HCl). Without being bound by any particular theory, stripping results in an exchange of ions (H⁺ ↔ Li⁺) and the release of sequestered lithium from the organic phase into the acidified aqueous. The extracted lithium can quantified according to any technique known in the art.

In some embodiments of the present method, the one or more compounds have a selectivity coefficient for lithium ion of from 1 to 10, e.g., a selectivity coefficient of 1, a selectivity coefficient of 2, a selectivity coefficient of 3, a selectivity coefficient of 4, a selectivity coefficient of 5, a selectivity coefficient of 6, a selectivity coefficient of 7, a selectivity coefficient of 8, a selectivity coefficient of 9, or a selectivity coefficient of 10. In some embodiments, the one or more compounds used in the present method have a selectivity coefficient greater than about 1. In some embodiments, the one or more compounds used in the present method have a selectivity coefficient greater than about 3. In some embodiments, the one or more compounds used in the present method have a selectivity coefficient greater than about 5. In some embodiments, the one or more compounds used in the present method have a selectivity coefficient greater than about 7. In some embodiments, the one or more compounds used in the present method have a selectivity coefficient for lithium ion of greater than about 10.

In some embodiments, the one or more compounds of the present method have an extraction capacity of a least about 3 mg Li/g of compound from a LiCl salt solution. In some embodiments, the one or more compounds of the present method have an extraction capacity of a least about 6 mg Li/g of compound from a LiCl salt solution. In some embodiments, the one or more compounds of the present method have an extraction capacity of a least about 9 mg Li/g of compound from a LiCl salt solution. In some embodiments, the one or more compounds of the present method have an extraction capacity of a least about 12 mg Li/g of compound from a LiCl salt solution.

In some embodiments, the one or more compounds of the present method have an extraction capacity of at least about 1.1 mg Li/g of compound from a geothermal brine solution. In some embodiments, the one or more compounds of the present method have an extraction capacity of a least about 2.2 mg Li/g of compound from a geothermal brine solution. In some embodiments, the one or more compounds of the present method have an extraction capacity of a least about 3.3 mg Li/g of compound from a geothermal brine solution. In some embodiments, the geothermal brine solution is a Salton Sea brine solution or Synthetic Chile brine solution.

### Oligomeric Extractants

Polymerizable functionalities can be added to the extractants discussed and one or more types of extractants be polymerized together with or without non-ligand monomers. Oligomeric extractants allow for adjustment of the physiochemical properties of the extractant and extractant solution such as viscosity, solubility, and capacity.

In some embodiments, the present disclosure provides a polymer of Formula (III), prepared by a process comprising polymerizing a compound of Formula (I-C3) and a compound of Formula (II):

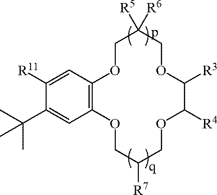

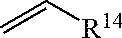

wherein R³, R⁴, R⁵, R⁶, R¹¹, p and q are as defined above in Formula (I) and Formula (I-B1);
R⁷ is H, —OH, -O-alkyl, -O-alkenyl, -O-alkynyl, -O-cycloalkyl, —(CH₂)ᵣOH, -(CH₂)ᵣO-alkyl, -O-alkylene-SiR¹³; —O—(CH₂)ₜC(O)OR⁸, —O—(CH₂)ₜS(O)₂OR⁸, —O—(CH₂)ₜS(O)₂N(R⁸)₂, —O—(CH₂)ₜP(O)₂(OR⁸)₂, or —O—(CH₂)ₜC(O)N(R⁹)₂, each of which is optionally substituted;
R¹³ is H, Cl, OH, alkyl, -O-alkyl, or aryl;
r is 1, 2, or 3;
t is independently 0, 1, or 2;
with the proviso that either R⁷ is -O-alkenyl or -O-alkylene-SiR¹³ or R¹¹ is -alkenyl; and
R¹⁴ is optionally substituted aryl or optionally substituted heteroaryl.

In some embodiments of Formula (I-C3), p and q are 0, and R³ and R⁴ are H.

In some embodiments of Formula (I-C3), R¹¹ is alkenyl. In some embodiments, the alkenyl is a C₂₋₁₂alkenyl. In some embodiments, the C₂₋₁₂alkenyl is vinyl.

In some embodiments of Formula (I-C3), R¹¹ is alkenyl and R⁷ is H, alkyl, —OH or -O-alkyl. In some embodiments, the alkyl is hexyl.

In some embodiments of Formula (I-C3), R⁷ is -O-alkenyl or -O-alkylene-SiR¹³. In some embodiments, R⁷ is -O-alkenyl. In some embodiments, the -O-alkenyl is -O(CH₂)ₖalkenyl, wherein k is an integer from 1-12. In some embodiments, the-O-alkenyl is —OCH₂CH═CH. In some embodiments, R⁷ is -O-alkylene-SiR¹³. In some embodiments, R¹³ is H, OH or halogen.

In some embodiments of Formula (I-C3), R⁷ is -O-alkenyl or -O-alkylene-SiR¹³ and R¹¹ is H, alkyl, haloalkyl, or cycloalkyl. In some embodiments, R⁷ is -O-alkenyl. In some embodiments, the -O-alkenyl is -O(CH₂)ₖalkenyl, wherein k is an integer from 1-12. In some embodiments, the-O-alkenyl is —OCH₂CH═CH. In some embodiments, R⁷ is -O-alkylene-SiR¹³ and R¹¹ is H, alkyl, haloalkyl, or cycloalkyl. In some embodiments, R¹³ is H, OH or halogen. In some embodiments, R¹¹ is H.

In some embodiments of Formula (I-C3), R¹⁴ is optionally substituted aryl. In some embodiments, the optionally substituted aryl is optionally substituted phenyl. In some embodiments, R¹⁴ is phenyl. In some embodiments, R¹⁴ is optionally substituted heteroaryl. In some embodiments, the optionally substituted heteroaryl is optionally substituted pyridinyl. In some embodiments, R¹⁴ is pyridinyl.

In some embodiments of Formula (I-C3), the lithium chelating is selected from the group consisting of 4-hydroxyl-bis(4′-t-butyl)dibenzo-14-crown-4 ether, 4,11-dihydroxyl-bis(4′-t-butyl)dibenzo-14-crown-4 ether, (4′-t-butyl)benzo-12-crown-4 ether, (4′-t-butyl)cyclohexyl-12-crown-4 ether, bis(4′-t-butyl)dibenzo-14-crown-4 ether, bis(4′-t-butyl)dicyclohexyl-14-crown-4 ether, 4-alkylhydroxyl-bis(4′-t-butyl)dibenzo-14-crown-4 ether, 4,11-dialkylhydroxyl-bis(4′-t-butyl)dibenzo-14-crown-4 ether, sym(4′-t-butyl)dibenzo-14-crown-4-oxyacetic acid ether, sym(4′-t-butyl)dibenzo-14-crown-4-oxysulfuric acid ether, sym(4′-t-butyl)dibenzo-14-crown-4-oxyphenylphosphonic acid ether, or sym(4′-t-butyl)dibenzo-14-crown-4-oxy-N-((trifluoromethyl)sulfonyl)acetamide ether.

In some embodiments of Formula (I-C3), one or more of the following groups is attached at one or more points along the polyether or polyamine linear and/or macrocyclic chains: phenyl, aromatic, linear or branched alkyl, cyclohexyl, ether, polyether, poly(ethylene oxide), poly(propylene oxide), amine, polyamine, phosphate, phosphite, carboxylic acid derivative, phosphonic acid derivative, sulfonic acid derivative, amino acid derivative, trifluoromethylsulfonyl carbamoyl, or other proton-ionizable group.

In some embodiments of Formula (I-C3), the polymer has the structural formula:

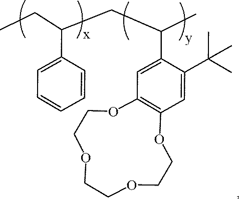

wherein x is an integer between 0 and 10 and y is an integer between 1 and 10.

In one embodiment, a vinyl group is attached to one of the atoms of the macrocycle. More specifically, the vinyl group is attached to a carbon, nitrogen, phenyl, or aromatic group. In a preferred embodiment is sym(4′-t-butyl)dibenzo-14-crown-4-oxyallyl ether. In a more preferred embodiment is (4′-t-butyl-3′-vinyl)benzo-12-crown-4 ether.

In another embodiment, a vinyl group is attached with a spacer to one or more atoms in the macrocycle. The spacer can consist of an alkyl, ether, polyether, thioether, amine, polyamine, phenyl, and/or aromatic constituents. In a preferred embodiment is sym(4′-t-butyl)dibenzo-14-crown-4-oxyalkylallyl ether, and sym(4′-t-butyl)dibenzo-14-crown-4-alkylallyl ether.

In one embodiment, a silane group is attached to one of the atoms of the macrocycle. More specifically, the silane group is attached to a carbon, nitrogen, phenyl, or aromatic group In a preferred embodiment is sym(4′-t-butyl)dibenzo-14-crown-4-(oxydialkoxy silane) ether.

In another embodiment, a silane group is attached with a spacer to one or more atoms in the macrocycle. The spacer can consist of an alkyl, ether, polyether, thioether, amine, polyamine, phenyl, and/or aromatic constituents. In a preferred embodiment is sym(4′-t-butyl)dibenzo-14-crown-4--(oxyalkyldialkoxy silane) ether.

### Polymeric Bead Sorbents

Extractants with polymerizable functionalities are capable of forming solid polymeric sorbents for the sequestration of lithium. In this embodiment one or more of the extractants containing polymerizable groups, such as a vinyl group, may or may not be mixed with one or more non-ligand monomers, one or more crosslinking monomers, and an initiator to be polymerized in a bulk, suspension, emulsion, or reverse-phase emulsion polymerization. These processes may use a radical, controlled radical, anionic, cationic, condensation, addition, or step polymerization mechanism.

In one embodiment, the polymeric bead sorbents are made from a polymerizable mixture containing, optionally, one or more ligand monomers, one or more non-ligand monomers, and one or more crosslinking monomers. In a preferred embodiment alkoxysilanes PDMS and sym(4′-t-butyl)dibenzo-14-crown-4--(oxyalkyldialkoxy silane) ethers can optionally be mixed together and undergo a bulk polymerization through a hydrolysis and condensation mechanism. In a more preferred embodiment styrene, divinylbenzene, sym(4′-t-butyl)dibenzo-14-crown-4-oxyallyl ether, and (4′-t-butyl-3′-vinyl)benzo-12-crown-4 ether can, optionally, be mixed together and undergo a suspension polymerization.

### Solid Sorbent

Solid sorbents can alternatively be made from a starting solid support and the macrocyclic ligand can be coated, adsorbed, or chemically attached to the surface of the solid support. The use of a solid support can have many advantages including: cost, reduced manufacturing time, unique synthetic routes, increased surface area and pore structure, additional physical properties related to the solid support chemical composition.

In one embodiment, one or more of the polymerizable extractants and, optionally, a non-ligand monomer, and a crosslinker are polymerized “around” a solid support completely or partially encasing it leaving the surface of the material with active sites for lithium adsorption. In a preferred embodiment the solid support is glass, alumina, magnetic particles, or other inorganic. In a more preferred embodiment the solid support is silica, or a lithium ion sieve.

In another embodiment, the extractant is chemically attached to the surface of the solid support. In a preferred embodiment the extractant is functionalized with a chlorosilane, alkoxysilane, or phosphate and attached to the metal hydroxide groups on the surface of the solid support. The solid support consist of silica, alumina, LIS, or other metal oxides.

### Membranes

Membranes can be made using similar techniques to the polymeric beads and solid sorbents with the simple alteration of making a material with a fiber morphology instead of a particle morphology. From a starting fibrous solid support the macrocyclic ligand can be coated, adsorbed, or chemically attached to the surface of the solid support. The use of a solid support can have many advantages including: cost, reduced manufacturing time, unique synthetic routes, increased surface area and pore structure, additional physical properties related to the solid support chemical composition.

In one embodiment, one or more of the polymerizable extractants and, optionally, a non-ligand monomer, and a crosslinker are polymerized “around” a fibrous solid support completely or partially encasing/coating it leaving the surface of the material with active sites for lithium adsorption. In a preferred embodiment the fibrous solid support is made from a polymeric, ceramic, or inorganic materials, or a mixture thereof. In a more preferred embodiment the fibrous solid support is made from silica, alumina, titania, zirconia, silicon carbide, carbatious or graphitic materials, cellulose or cellulose derivative, polyethylene, polypropylene, cellulose, nitrocellulose, cellulose esters, polysulfone, polyethersulfones, polyacrilonitrile, polyamide, polyimide, polyethylene, polypropylene, polytetrafluoroethylene, polyvinylidene fluoride, polyvinylchloride, or composites thereof. In a more preferred embodiment the solid support is silica, or a lithium ion sieve material.

In another embodiment, the extractant is chemically attached to the surface of the fibrous solid support. In a preferred embodiment the fibrous solid support is an inorganic, ceramic, metal oxide, or polymeric material, or a composite of one or more of these materials. In a more preferred embodiment the extractant is functionalized with a chlorosilane, alkoxysilane, or phosphate and attached to the metal hydroxide groups on the surface of the fibrous solid support. The fibrous solid support consist of silica, alumina, titania, zirconia, LIS, or other metal oxides.

In another embodiment, the membrane fibers are made from a polymerizable mixture containing, optionally, one or more ligand monomers, one or more non-ligand monomers, and one or more crosslinking monomers. In a preferred embodiment alkoxysilanes PDMS and sym(4′-t-butyl)dibenzo-14-crown-4--(oxyalkyldialkoxy silane) ethers can, optionally, be mixed together and made into a fibrous membrane. In a more preferred embodiment styrene, divinylbenzene, sym(4′-t-butyl)dibenzo-14-crown-4-oxyallyl ether, and (4′-t-butyl-3′-vinyl)benzo-12-crown-4 ether can optionally be mixed together and made into a fibrous membrane.

### Extractions

Lithium extraction is broken down into two processes, extraction and elution. Extraction consists of selectively removing a target molecule from a source phase, in this case lithium, to an extraction phase. Elution entails releasing lithium from the extraction phase into the elution phase for final processing. The extraction and elution process can be separate or coupled stages depending on the design of the system. The source phase is a lithium containing solution, generally an aqueous solution, and may contain contaminants in varying concentrations, such as metal ions, dissolved silicates, and dissolved organics. The extraction phase can come in several different forms that depend on the type of extraction technique used such as liquid/liquid extraction, solid sorbent column filtration, membrane filtration, nanofiltration, liquid supported membrane extraction, ion-exchange, and emulsion liquid membrane extraction. The extraction phase can consist of an organic phase with dissolved extractants and other promoters, this would be used in a liquid/liquid extraction setup, a solid sorbent which is contacted with the source phase and then filtered out, such as in a solid sorbent filtration column setup, as a membrane which can consist of solid components and/or a liquid organic phase which can have extractants attached to the surface of the membrane or dissolved in the organic phase. The elution phase consists of an eluent that is contacted with the extraction phase and releases the lithium into the eluent. The eluent consists of an aqueous acid solution and optionally other dissolved ions to promote the release of lithium. The lithium is released by an ion-exchange mechanism, generally lithium, exchanged for hydrogen or another cationic species.

### Source Phase

In one embodiment, the source phase is a natural brine, a dissolved salt flat, seawater, concentrated seawater, desalination effluent, a concentrated brine, a processed brine, a geothermal brine, liquid from an ion exchange process, liquid from a solvent extraction process, a synthetic brine, leachate from ores, leachate from minerals, leachate from clays, leachate from recycled products, leachate from recycled materials, or combination thereof.

In another embodiment, the source phase has a lithium concentration from 100,000 ppm - 0.001 ppm. More preferably greater than 100 ppm, and more preferably greater than 500 ppm.

In another embodiment, the molar ratio of any contaminating or interfering species is less than 100,000:1. More preferably less than 10,000:1, and more preferably less than 1,000:1.

In another embodiment, the contaminating species consist of metal ions from the alkaline, alkali earth, and transition metals and or silicate species. More specifically Na, K, Rb, Cs, Mg, Ca, Sr, Ba, B, Si, Mn, Fe, Zn, Pb, As, Cu, Cd, Ti, Sb, Ag, V, Ga, Ge, Se, Be, Al, Ti, Co, Ni, Zr, and combinations thereof.

In another embodiment, the source phase consists of high concentrations of common water soluble anions. More specifically Cl, SO₄, NO₃, and combinations thereof.

In another embodiment, the source phase may contain up to 50% total dissolved solids (TDS). More preferably less than 35% TDS, and even more preferably less than 15% TDS.

In another embodiment, the source phase may be at elevated temperature less than 500° C. More preferably less than 300° C., even more preferably less than 110° C., and yet more preferably ambient temperatures.

In another embodiment, the source phase may be at elevated pressure less than 500 PSIG. More preferably less than 50 PSIG and even more preferably at atmospheric pressure.

In another embodiment, the source phase has a pH of 0-14. More preferably the pH is greater than 5.0, and even more preferably the pH is greater than 7.0, and yet more preferably the pH is greater than 10.0.

### Extraction Phase

Liquid/liquid extraction configurations contacts the source phase with an organic phase for a certain residence time and may be agitated to increase interfacial surface area.

In one embodiment, the extraction phase is comprised of an organic phase which may contain organic solvents, diluents, ionic liquids, phosphates, organic acids, small molecule macrocyclic extractants, oligomeric macrocyclic extractants, polymeric macrocyclic extractants, suspended particles, suspended lithium ion sieves, surfactants, micelles, suspensions, emulsions, and a combination thereof.

In another embodiment, the diluent contains, linear or branched alkanes, aromatics, siloxanes, large alkyl chain alcohols, ketones, chlorinated hydrocarbons, fluorinated hydrocarbons, sulfonated hydrocarbons, or mixtures thereof.

In another embodiment, residence times are less than 24 hours. More preferably less than 1 hour, even more preferably less than 30 minutes, and yet more preferably less than 5 minutes.

Emulsion liquid membranes (ELMs) are prepared by dispersing an inner receiving phase in an immiscible liquid membrane phase to form an emulsion. The liquid membrane phase is organic thus forming water-in-oil emulsions. The formation of stable water-in-oil ELMs is based on a number of factors including: surfactant concentration, organic viscosity, and volume ratios of the various phases. The water-in-oil emulsion is formed by mixing the receiving phase with the organic phase. The emulsion is then transferred into the source phase, allowing the lithium to transfer from the outer source phase, across the organic phase, and into the inner receiving phase. This process essentially couples the extraction and elution process. There is a delicate balance that is struck between making the emulsions strong enough to resist the shear stress during agitation with the source phase, and isolating the emulsion and breaking it to release the receiving phase.

In one embodiment, the liquid membrane phase is comprised of an organic phase which may contain organic solvents, diluents, ionic liquids, phosphates, organic acids, small molecule macrocyclic extractants, oligomeric macrocyclic extractants, polymeric macrocyclic extractants, suspended particles, suspended lithium ion sieves, surfactants, micelles, suspensions, emulsions, and a combination thereof to facilitate the transport of lithium across the liquid membrane.

In another embodiment, the surfactants can be cationic, non-ionic, anionic, polymeric, small molecule, and combinations thereof.

In another embodiment, the diluent contains, linear or branched alkanes, aromatics, siloxanes, large alkyl chain alcohols, ketones, chlorinated hydrocarbons, fluorinated hydrocarbons, sulfonated hydrocarbons, or mixtures thereof.

In another embodiment, the receiving phase, is the same as the eluent, is an aqueous acid solution containing hydrochloric acid, sulfuric acid, phosphoric acid, hydrobromic acid, chloric acid, perchloric acid, nitric acid, formic acid, acetic acid, carbonic acid, and combinations thereof, including derivatives thereof.

In another embodiment, the acid concentration is less than about 18 M. More preferably less than about 2 M, and even more preferably less than about 1 M.

In another embodiment, residence times are less than 24 hours. More preferably less than 1 hour, even more preferably less than 30 minutes, and yet more preferably less than 5 minutes.

Solid sorbents are a solid extraction phase that contains many selective binding sites and are directly put in contact with the source phase. The solid/liquid interaction is characterized by surface area, wettability of the sorbent surface, and residence time. The solid sorbents can be in the form of powders, beads, granules, fibers, crushed material, irregular shaped particles, or combinations thereof. Solid sorbents are easily separated by filtrations, centrifugation, or other gravimetric means. The core of solid sorbents can even be made of magnetic materials and be manipulated with external magnetic fields. These materials can be used in continuous flow column or batch configurations.

In one embodiment, the solid sorbent is made by polymerizing an extractant, containing a polymerizable functionality, with optionally, one or more non-ligand monomers, and crosslinkers. More preferably the extractant is a macrocyclic ligand containing a vinyl functionality.

In another embodiment, the solid sorbent is made by using a premade solid support and coating or encasing the solid support with a polymerizable reaction mixture that contains one or more vinyl functionalized extractants, one or more non-ligand monomer, and one or more crosslinker.

In another embodiment, the solid support is made of silica, alumina, titania, iron oxide, manganese oxide, glass, metal oxide, polystyrene, or other inorganic or polymeric material.

In another embodiment, the solid sorbent is made by entrapping extractants in a polymer matrix by using a premade solid support and coating or encasing the solid support with a polymerizable reaction mixture that contains one or more non-monomer extractants, one or more non-ligand monomer, and one or more crosslinker.

In another embodiment, the solid sorbent is made by entrapping extractants in a polymer matrix by using a premade solid support and coating or encasing the solid support with a solution of one or more dissolved polymers, one or more extractants, and optionally, one or more phase transfer agents.

In another embodiment, the solid sorbent is made by chemically attaching the extractant or functionalizing the surface of the solid support with the extractant. More preferably the extractant is macrocyclic and attached to a metal oxide surface with a silane or phosphate linkage.

Membranes act as a physical barrier that separates the source phase and the elution phase or acts as an immobilized extraction phase that allows the source phase to flow through it.

In one embodiment, the extractants are chemically attached to the membrane.

In another embodiment, the membrane is coated or encased in a polymeric material that may have the extractant chemically incorporated into its matrix, or have the extractant entrapped in the polymer matrix.

In another embodiment, the source phase is flowed through the membrane and the lithium is bound to the membrane.

In another embodiment, the source phase is flowed over the membrane and the lithium is bound to the membrane.

In another embodiment, the source phase and elution phase is separated by the membrane and lithium is transported from the source phase to the elution phase.

In supported liquid membranes the extraction phase consists of a physical membranes that contain an adsorbed organic phase to facilitate loading the membrane with extractants and faster transport. Spiral wound and hollow fiber geometries increase the surface area of liquid membrane modules, improving overall efficiency.

In one embodiment, the extraction phase is comprised of an organic phase which may contain organic solvents, diluents, ionic liquids, phosphates, organic acids, small molecule macrocyclic extractants, oligomeric macrocyclic extractants, polymeric macrocyclic extractants, and a combination thereof.

In another embodiment, the physical membrane may be made from a polymeric, inorganic, or bio-based material.

### Elution Process

The elution process used to recover lithium is undertaken by contacting the eluent with the extraction phase, producing a concentrated lithium solution. The elution may happen in a batch or continuous flow process, happen at elevated temperatures, and/or consist of acid solutions and/or other dissolved cationic species.

In one embodiment, the eluent is an aqueous acid solution containing hydrochloric acid, sulfuric acid, phosphoric acid, hydrobromic acid, chloric acid, perchloric acid, nitric acid, formic acid, acetic acid, carbonic acid, and combinations thereof, including derivatives thereof.

In another embodiment, the acid concentration is less than about 18 M. More preferably less than about 2 M, and even more preferably less than about 1 M.

In another embodiment, the elution is done at elevated temperatures less than 110° C. More preferably less than 60° C., and even more preferably at ambient temperatures.

The ion-exchange mechanism utilized by the materials described herein is reversible and the materials are designed for reuse and to have an extended lifespan.

In one embodiment, the extraction phase is used for more than 1 cycle. More preferably more than 50 cycles, even more preferably more than 100 cycles, and yet more preferably more than 300 cycles.

### EXAMPLES

### Example 1: Preparation of Small Molecule Extractants

### Exemplary Synthesis of Extractants:

Exemplary Synthesis of (4′-t-butyl)benzo-12-crown-4 ether (16):

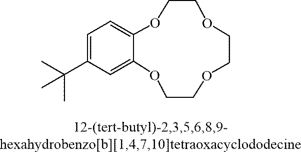

4-t-butylcatechol (45 g, 271 mmol) and bis(1-chloroethoxy)ethane (51 g, 273 mmol) were dissolved in 1-butanol (1 L) in a 2 L round bottom flask equipped with a stir bar, condenser, and nitrogen inlet. A solution of sodium hydroxide (50 mL, 11 M) was added to the reaction solution. The reaction was purged with nitrogen then heated to reflux with stirring under nitrogen. The reaction was run for 24 hours then cooled to room temperature. The reaction solution was filtered and the solvent removed by vacuum distillation to yield the crude product.

Compound 16: ¹H NMR CDCl₃ 400 MHz: δ 1.28 (s, 9H), 3.61-3.89 (m, 8H), 4.10-4.25 (m, 4H), 6.75-7.02 (m, 3H)

Exemplary Synthesis of bis(4′-t-butyl)dibenzo-14-crown-4 ether (17):

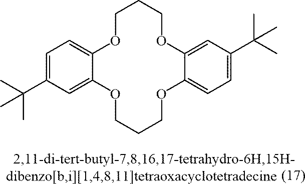

4-t-butylcatechol (16.62 g, 100 mmol) and lithium hydroxide (4.8 g, 200 mmol) were dissolved in 1-butanol (120 mL) in a 250 mL 3-neck round bottom flask equipped with a stir bar, addition funnel, condenser, and nitrogen inlet. After purging the reaction was purged with nitrogen the reaction was heated to reflux under nitrogen. During the heating ramp the first aliquot of 1,3-dibromopropane (10.1 g, 50 mmol) was added dropwise to the reaction solution. After the reaction reached reflux and the addition of the first aliquot of 1,3-dibromopropane was complete the reaction was let reflux for 1 hour. After the hour of reflux a second aliquot of 1,3-dibromopropane (10.1 g, 50 mmol) was added dropwise at reflux. The reaction temperature was held for 12 hours and then cooled to room temperature. The reaction solution was filtered and the solvent removed by vacuum distillation to yield the crude product.

Compound 17: ¹H NMR CDCl₃ 400 MHz: δ 1.28 (s, 18H), 2.32 (m, 4H), 4.25 (m, 8H), 6.78-6.93 (m, 4H), 7.01 (s, 2H)

Exemplary Synthesis of bis(4′-t-butyl)dibenzo-14-crown-4 ether-oxysulfonic acid (18):

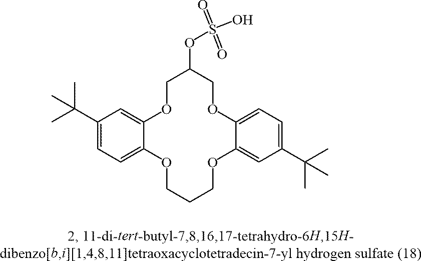

4-t-butylcatechol (16.62 g, 100 mmol) and lithium hydroxide (4.8 g, 200 mmol) were dissolved in 1-butanol (120 mL) in a 250 mL 3-neck round bottom flask equipped with a stir bar, addition funnel, condenser, and nitrogen inlet. After purging the reaction was purged with nitrogen the reaction was heated to reflux under nitrogen. During the heating ramp the first aliquot of 1,3-dibromopropane (10.1 g, 50 mmol) was added dropwise to the reaction solution. After the reaction reached reflux and the addition of the first aliquot of 1,3-dibromopropane was complete the reaction was let reflux for 1 hour. After the hour of reflux, a second aliquot of epichlorohydrin (4.63, 50 mmol) was added dropwise at reflux. The reaction temperature was held for 12 hours and then cooled to room temperature. The reaction solution was filtered and the solvent removed by vacuum distillation to yield the crude product. After the product was cleaned up, as specified by the procedure below, 10.0 g (22 mmol) was dissolved in THF in a 250 mL round bottom flask, equipped with a stirbar and purged with nitrogen. Chlorosulfonic acid (2.56 g, 22 mmol) was added dropwise over a few minutes under nitrogen with stirring. The chlorosulfonic acid reacted vigorously with the reaction solution and produced substantial fizzing. Efforts to reduce the rate of addition resulted in degradation of the chlorosulfonic acid reagent. The solvent was distilled off and the product cleaned by the procedure stated below.

Compounds of the present disclosure prepared in a similar manner are as follows:

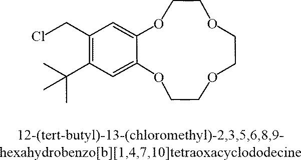

Compound 19: ¹H NMR CDCl₃ 400 MHz: δ 1.28 (s, 9H), 3.55-3.93 (m, 10H), 4.08-4.22 (m, 4H), 6.75-7.02 (m, 2H)

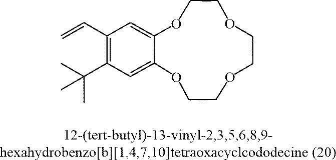

Compound 20: ¹H NMR CDCl₃ 400 MHz: δ 1.28 (s, 9H), 3.61-3.89 (m, 8H), 4.10-4.25 (m, 4H), 5.18 (d, 1H), 5.38 (d, 1H), 6.75-7.02 (m, 3H)

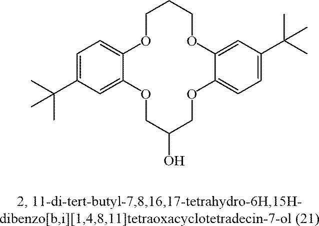

Compound 21: ¹H NMR CDCl₃ 400 MHz: δ 1.28 (s, 18H), 1.78 (br, 1H), 2.32 (m, 2H), 3.82 (m, 1H), 4.25 (m, 8H), 6.78-7.01 (m, 6H)

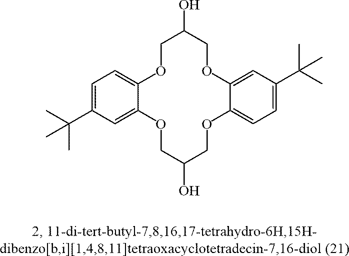

Compound 21: ¹H NMR CDCl₃ 400 MHz: δ 1.28 (s, 18H), 2.19 (br, 2H), 3.82 (m, 2H), 4.01-4.48 (m, 8H), 6.78-7.01 (m, 6H)

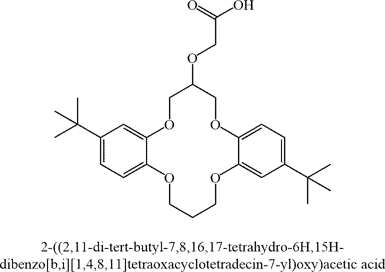

Compound 8: ¹H NMR CDCl₃ 400 MHz: δ 1.28 (s, 18H), 2.32 (m, 2H), 3.82 (m, 1H), 4.25 (m, 8H), 5.24 (s, 2H), 6.78-7.01 (m, 6H)

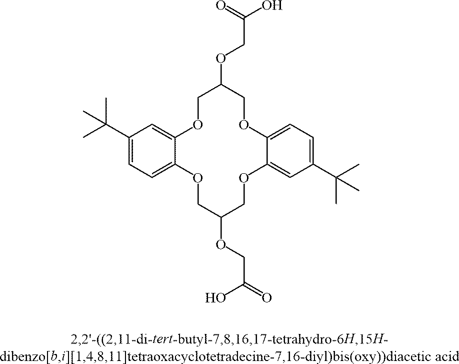

Compound 11: ¹H NMR CDCl₃ 400 MHz: δ 1.28 (s, 18H), 3.82 (m, 2H), 4.01-4.48 (m, 8H), 5.24 (s, 2H), 6.78-7.01 (m, 6H)

### Post-Reaction Crude Product Cleanup

The crude product was cleaned by dissolving in diethyl ether and washing with 100 mL of 1 M HCl x2 and 100 mL of DI water x3 or until the discarded aqueous phase has a neutral pH. The organic phase is then dried over anhydrous magnesium sulfate, and optionally filtered through a short bed of silica gel, then the product is crystalized by slow evaporation or the solvent is removed via vacuum distillation to yield the final product.

### Example 2: Preparation of Oligomeric Extractants

Any of the types of extractants described in example 1 can be functionalized into a ligand monomer by attaching a vinyl group to the benzene ring. An exemplary reaction is described.

### Chloromethylation

To a 50 mL round bottom flask fitted with a stir bar and nitrogen inlet, was added 10 g of product, from example 1, 1.8 g paraformaldehyde, and 15 mL concentrated HCl. The reaction mixture was purged with nitrogen and heated to 55° C. for 36 hours. The reaction mixture was extracted x3 with chloroform. The organic phases were combined and washed x2 with DI water or until the discarded aqueous phases had a neutral pH. The product phase was dried over anhydrous magnesium sulfate, filtered and the solvent vacuum distilled off.

### Vinyl Formation

5 g (15.2 mmol) of the chloromethylated product and triphenylphosphine (4.19 g, 16 mmol) was dissolved in 30 mL of DMF and added to a 100 mL round bottom flask equipped with a stirbar and condenser. The reaction was refluxed for 3 hours and then cooled to room temperature. 50 mL of 40% formaldehyde solution in water and 16 mL of 12.5 M NaOH was added to the reaction mixture and the reaction was stirred at <40° C. for 2 hours. The reaction solution was filtered and the solvent vacuum distilled off to yield the crude product. The crude product was purified as stated previously in the post reaction cleanup procedure.

### Example 3: Preparation of Macroreticular Beads

### Exemplary Suspension Polymerization:

### Preparation of Aqueous Phase

Polyvinyl alcohol (PVOH, average Mw 89,000-98,000, 99+% hydrolyzed, 10.26 g) is dissolved in water (540 mL) through gentle heating to 80° C. 4.42 g of boric acid is dissolved in 135 mL in water and slowly added when the PVOH cools to 50° C.

### Preparation of The Organic Phase And Polymerization

5 g of the ligand monomer is combined with 48.75 mL of 2-ethylhexanol and 1.25 mL of xylenes in a 100 mL Erlenmeyer flask equipped with a stir bar and allowed to stir until fully dissolved. 35.88 mL of styrene and 13.68 mL of divinylbenzene are combined with the solution of monomer ligand, and allowed to stir, covered with a septum, under ambient conditions. 0.5 g of AIBN is added to the solution and dissolved completely. When dissolved, the solution is added to an addition funnel and degassed until the reaction temperature reaches 75° C. When the temperature reaches 80° C. the solution is added to the aqueous phase at a rate of 1 mL/s. The reaction is allowed to proceed, with continuous agitation for approximately 8 hours.

### Post-Reaction Bead Cleanup

Upon completion of the reaction, the beads are recovered from the aqueous by filtration. The beads are then soaked in deionized water (200 mL) for 10 minutes then filtered. Soaking in deionized water and filtration is repeated two times. The beads are washed twice in methanol, and twice in acetone. If desired, the beads can be fractionated by size using the appropriate mesh sieves. The beads can then be stored in water indefinitely at a temperature of 5 to 50° C.

### Example 4: Recovery of Lithium From LiCl Brine Solution

General method for extracting Li from LiCl brine solution: 150 mg of extractant (e.g., (t-butyl)benzyl-12-crown-4 ether) dissolved in 15 mL of diluent (e.g., 1-ethylhexanol) was contacted with 15 mL of an aqueous 250 ppm LiCl solution at pH 5.5 and shaken from 30 seconds to 24 hours (note: complete extraction occurs after about 5 minutes) at 60° C. Extracted Li was calculated by comparing the metal concentration in the initial solution (feed) and the metal concentration in the solution after treatment (barren). The concentration of the metal ions in solution was determined by inductively coupled plasma mass spectrometry (ICP-MS).

Parameters for evaluating lithium capacity in LiCl solution:
Aqueous phase – 250 ppm LiCl at pH 5.5 ± 0.3
Diluents - multiple diluents tested (kerosene, paraffin, 1-octanol, 2-ethyl-1-hexanol
Organic solution (O)/Aqueous solution (A) – 1:1 by volume
Organic phase preparation – 0.15 g of extractant dissolved in 15 mL of diluent in a 40 mL glass sample vial. Dissolved at 60° C. with agitation (shaker box)
Extraction – 15 mL of aqueous phase (preheated) added to the organic phase (preheated) and extracted at 60° C. with agitation (shaker box) for 4 hours.
Analysis – 3 mL sample of the aqueous phase stock solution and the aqueous phase after extraction. Lithium analysis by ICP-MS.

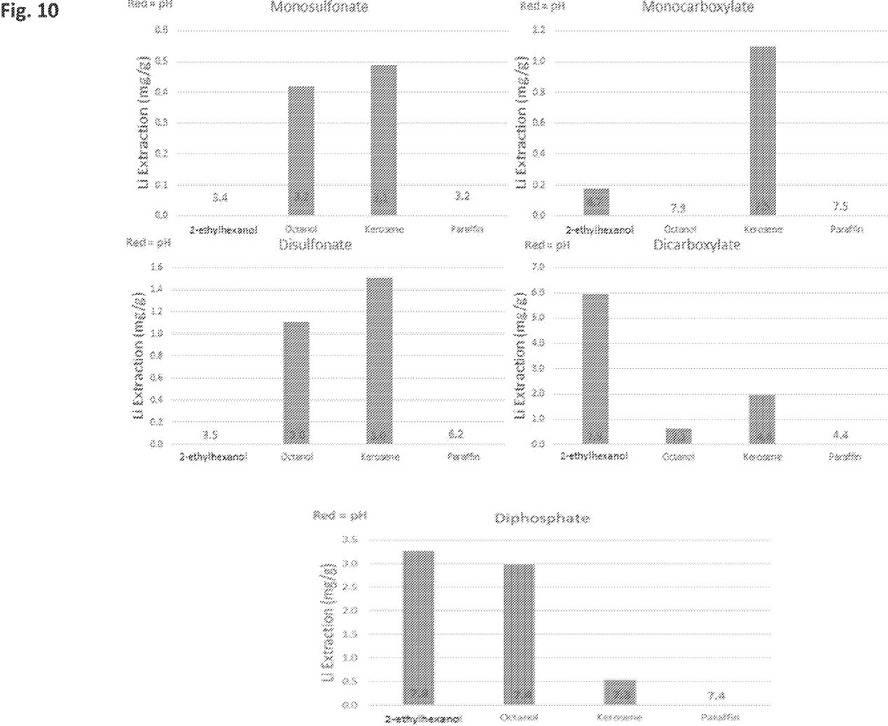

Solvent Effects: FIG. 10 shows the effect of diluent on lithium extraction from an LiCl brine solution for a series of extractants (monocarboxylate 8, monosulfate 10, dicarboxylate 11, and disphosphate 12, disulfate 13) comprising different chelating functional groups. It was found that dicarboxylate extractant 11 in 2-ethyl-1-hexanol was able to remove 6 mg of lithium/g of extractant from a LiCl brine solution. Also of note was the performance of the sulfate-based materials 10 and 13, as those extractions resulted in final pH values that were generally lower than the other extractants tested.

### Summary of Lithium Recovery Data From LiCl Brine Solution

TABLE 1

Li Extraction Capacity from 250 ppm LiCl Brine Solution

| Sample | Metals | Extractant | Aq. Vol | Feed | Barren | Extraction | | | Recovery | Temp | pH |
|---|---|---|---|---|---|---|---|---|---|---|---|
| | | (g) | (L) | (ppm) | (ppm) | (ppm) | (mg) | (mg/g) | (%) | (C) | |
| 2 | Li | 0.15 | 0.015 | 240.798 | 181.205 | 59.6 | 0.9 | 6.0 | 24.7 | 60 | 7.8 |
| 57 | Li | 0.15 | 0.015 | 242.9 | 210.3 | 32.6 | 0.5 | 3.3 | 13.4 | 60 | 7.8 |
| 61 | Li | 0.16 | 0.015 | 246.0 | 214.3 | 31.7 | 0.5 | 3.0 | 12.9 | 60 | 7.4 |
| 102 | Li | 0.16 | 0.015 | 247.137 | 221.088 | 26.0 | 0.4 | 2.4 | 10.5 | 66 | 8.7 |
| 48 | Li | 0.16 | 0.015 | 240.798 | 215.981 | 24.8 | 0.4 | 2.3 | 10.3 | 60 | 7.1 |
| 79 | Li | 0.15 | 0.015 | 244.616 | 222.029 | 22.6 | 0.3 | 2.3 | 9.2 | 60 | 6.5 |
| 96 | Li | 0.17 | 0.015 | 247.137 | 222.963 | 24.2 | 0.4 | 2.1 | 9.8 | 60 | 8.8 |
| 101 | Li | 0.15 | 0.015 | 247.137 | 226.266 | 20.9 | 0.3 | 2.1 | 8.4 | 65 | 3.9 |
| 112 | Li | 0.17 | 0.015 | 247.137 | 224.944 | 22.2 | 0.3 | 2.0 | 9.0 | 67 | 7.6 |

Sample Key for Table 1

| Sample No. | Compound No. | Organic Phase | Sample No. | Compound No. | Organic Phase |
|---|---|---|---|---|---|
| 2 | 11 | 2-ethylhexanol | 79 | 9 | 2-ethylhexanol |
| 57 | 12 | 2-ethylhexanol | 96 | Commercial Ionic Liquid | |
| 61 | 12 | octanol | 101 | Cyanex 272 (Commercial Extractant) | |
| 102 | Commercial Ionic Liquid | | 112 | 2 | 2-ethylhexanol |
| 48 | 19 | 2-ethylhexanol | | | |

Results: Batch testing of the diluent/extractant systems at a 1% w/w loadings were used to screen samples and minimize the amount of extractant required. From 250 ppm LiCl brine, several different extractants (i.e., compounds 2, 9, 11, 12, and 19) achieved respectable extraction capacities, with 6 mg Li/g extractant being the largest quantity extracted using dicarboxylate 2 (Table 1).

### Example 5: Recovery of Lithium From Salton Sea Brine

Salton Sea Brine is a geothermal brine that contains various amounts of dissolved metals. The composition of the Salton Sea brine used in the present study is shown in Table 2.

TABLE 2

Composition of metal ions in Salton Sea Brine at pH 5.4

| | Composition | | | | | Mass Ratios | | | |
|---|---|---|---|---|---|---|---|---|---|
| | Li | Na | Mg | K | Ca | Na/Li | Mg/Li | K/Li | Ca/Li |
| Salton Sea Brine Sample | ppm | ppm | ppm | ppm | ppm | | | | |
| pH 5.4 | 248 | 56612 | 57 | 18148 | 18288 | 228.6 | 0.2 | 73.3 | 73.9 |

General method of extracting lithium from Salton Sea Brine: 150 mg of extractant (e.g., (t-butyl)benzyl-12-crown-4 ether) dissolved in 15 mL of diluent (e.g., 1-ethylhexanol) was contacted with 15 mL of an aqueous 250 ppm Salton Sea brine solution at pH 5.5 and shaken from 30 seconds to 24 hours (note: complete extraction occurs after about 5 minutes) at 60° C. Extracted Li was calculated by comparing the metal concentration in the initial solution (feed) and the metal concentration in the solution after treatment (barren). The concentration of the metal ions in solution was determined by inductively coupled plasma mass spectrometry (ICP-MS).

Parameters for evaluating lithium capacity in Salton Sea Brine under batch conditions (Table 3):
Aqueous phase - Salton Sea Brine at pH 5.5 ± 0.3
Diluent - 2-ethyl-1-hexanol, octanol, mineral oil, kerosene
Organic solution (O)/Aqueous solution (A) - 1:1 by volume
Organic phase preparation - 0.15 g of extractant dissolved in 15 mL of diluent in a 40 mL glass sample vial. Dissolved at 60° C. with agitation (shaker box)
Extraction - 15 mL of aqueous phase (preheated) added to the organic phase (preheated) and extracted at 100° C. under reflux with stirring for 4 hours.
Stripping - 5 mL of 1 M HCl aqueous phase added to 5 mL of the organic phase and agitated at 60° C. (orbital shaker) for 4 hours.
Analysis - 3 mL samples of the aqueous phase stock solution, the aqueous phase after extraction, and the stripping phase. Lithium analysis of aqueous phase before and after extraction, full metal analysis of stripping phase. All samples analyzed by ICP-MS.

Table 3 includes the results from various extractant/diluent systems at 1% w/v loadings. Lithium was extracted in accordance with the flow chart provided in FIG. 8. The amount of lithium extracted and the percent recovery are provided. pH ranges from 2.1 to 7.1 for the aqueous phase.

TABLE 3

Li Extraction Capacity from Salton Sea Brine (Barren vs. Feed)

| Sample | Metals | Extratant | Aq. Vol | Feed | Barren | Extraction | | | Recovery | Temp | pH |
|---|---|---|---|---|---|---|---|---|---|---|---|
| | | (g) | (L) | (ppm) | (ppm) | (ppm) | (mg) | (mg/g) | (%) | (C) | |
| 89 | Li | 0.15 | 0.015 | 284.0 | 261.9 | 22.1 | 0.3 | 2.2 | 7.8 | 60 | 5.2 |
| 116 | Li | 0.15 | 0.015 | 293.7 | 272.1 | 21.6 | 0.3 | 2.2 | 7.4 | 60 | 7.1 |
| 84 | Li | 0.18 | 0.015 | 284.0 | 263.5 | 20.5 | 0.3 | 1.7 | 7.2 | 60 | 4.2 |
| 109 | Li | 0.15 | 0.015 | 293.7 | 276.7 | 17.0 | 0.3 | 1.7 | 5.8 | 60 | 5.3 |
| 105 | Li | 0.15 | 0.015 | 282.5 | 266.6 | 15.9 | 0.2 | 1.6 | 5.6 | 60 | 5.6 |
| 103 | Li | 0.15 | 0.015 | 282.5 | 266.7 | 15.8 | 0.2 | 1.6 | 5.6 | 60 | 5.3 |
| 93 | Li | 0.16 | 0.015 | 282.5 | 266.5 | 16.0 | 0.2 | 1.5 | 5.7 | 60 | 4.5 |
| 92 | Li | 0.18 | 0.015 | 282.5 | 264.6 | 17.9 | 0.3 | 1.5 | 6.3 | 60 | 3.8 |
| 124 | Li | 0.16 | 0.015 | 284.0 | 269.1 | 14.9 | 0.2 | 1.4 | 5.2 | 60 | 6.8 |
| 86 | Li | 0.18 | 0.015 | 284.0 | 269.2 | 14.8 | 0.2 | 1.2 | 5.2 | 60 | 5.6 |
| 80 | Li | 0.15 | 0.015 | 284.0 | 272.0 | 12.0 | 0.2 | 1.2 | 4.2 | 60 | 5.5 |
| 107 | Li | 0.16 | 0.015 | 282.5 | 273.0 | 9.5 | 0.1 | 0.9 | 3.4 | 60 | 2.1 |

Sample Key for Table 3 and Table 4 (below)

| Sample No. | Compound No. | Organic Phase | Sample No. | Compound No. | Organic Phase |
|---|---|---|---|---|---|
| 89 | 8 | 2-ethylhexanol | 93 | 8 | kerosene |
| 116 | 2 | 2-ethylhexanol | 92 | 11 | kerosene |
| 84 | 11 | octanol | 124 | 1 | 2-ethylhexanol |
| 109 | Commercial Tributylphosphate | | 86 | 15 (v=0) | 2-ethylhexanol |
| 105 | Commercial dibenzo 15-Crown-5 ether | | 80 | 16 | mineral oil |
| 103 | 14 (v=0) | mineral oil | 107 | 19 | 2-ethylhexanol |

Lithium Capacity Results: Testing barren vs. feed samples from Salton Sea brine extracted with the above samples produced lithium extraction capacities that were comparable to the LiCl brine results (Table3). Data was also obtained by analyzing the amount of lithium in the acid elution after treating the organic phase with aqueous acid (Table 4). These results show the first known successful liquid-liquid extraction of lithium from geothermal brines.

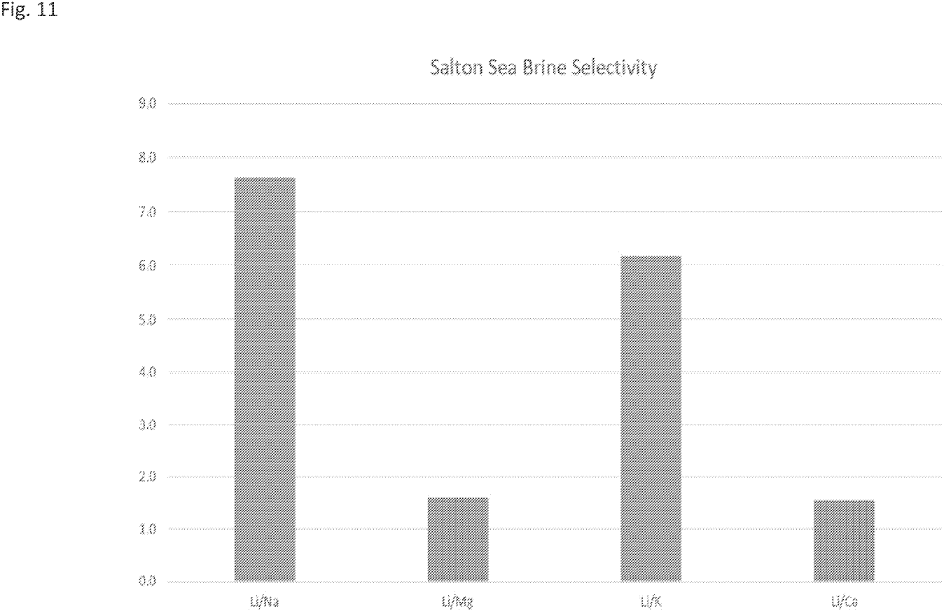

Lithium Selectivity Results: Selectivity is provided by comparing metal ion ratios in the eluted acidified aqueous solution (FIG. 11) to ion ratios in the feed solution (Table 2) for Salton Sea brine. FIG. 11 provides ratios for Li/Na, Li/Mg, Li/K, and Li/Ca after treating the brine with an extractant disclosed herein using the protocol described above. In each case, lithium was selectively extracted using the liquid-liquid extraction method described herein even though the concentration of Na, K, and Ca in Salton Sea brine is substantially higher than the concentration of Li. Thus, the data shows that liquid-liquid extraction using compounds of the present disclosure is able to successfully enrich the aqueous acidified solution with lithium from Salton Sea brine.

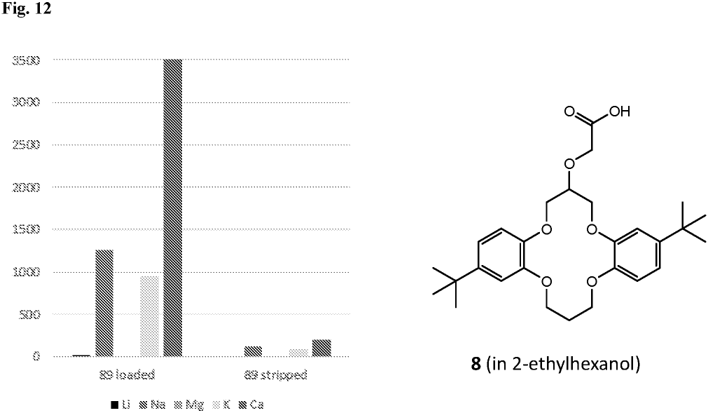

The effectiveness of the extraction is highlighted in FIG. 12 showing the digestion of the organic phase before (loaded) and after (stripped) elution. The organic phase containing Compound 8 that was used to extract lithium from the Salton Sea brine solution was stripped with (1 N HCl), which results in the transfer of metal ions into the aqueous phase. FIG. 12 shows the efficiency of this process as the organic phase after acidic water treatment (89 stripped) has very low concentrations of metal ions compared to the loaded phase prior to elution.

TABLE 4

Lithium Extraction Capacity from Salton Sea Brine in the Acidic Elution

| | | | | | | | | | | | | | | | | | | | barren vs. feed |
|---|---|---|---|---|---|---|---|---|---|---|---|---|---|---|---|---|---|---|---|
| Sample | Sol | Crown | Metals | | | | | | | | | | | Selectivity | | | | | C - samples |
| | | | Li {eluted from extractant) | Na | Mg | K | Ca | Li | Na | Mg | K | Ca | Total | Li | Na | Mg | K | Ca | Li |
| | L | g | ppm | ppm | ppm | ppm | ppm | mg/g | mg/g | mg/g | mg/g | mg/g | mg/g | (%) | (%) | (%) | (%) | (%) | ppm |
| R80 | 0.015 | 0.15 | 0.04 | 30.40 | 0.00 | 0.00 | 0.00 | 0.0 | 3.0 | 0.0 | 0.0 | 0.0 | 3.0 | 0.1 | 99.9 | 0.0 | 0.0 | 0.0 | 12 |
| R84 | 0.015 | 0.18 | 4.94 | 68.44 | 0.21 | 24.83 | 210.50 | 0.4 | 6.8 | 0.0 | 2.5 | 21.1 | 30.8 | 1.3 | 22.2 | 0.1 | 8.1 | 68.3 | 20.5 |
| R86 | 0.015 | 0.18 | 5.09 | 56.54 | 0.22 | 13.42 | 100.80 | 0.5 | 5.7 | 0.0 | 1.3 | 10.1 | 17.6 | 2.9 | 32.1 | 0.1 | 7.6 | 57.3 | 14.8 |
| R89 | 0.015 | 0.15 | 51.68 | 4362. 69 | 10.11 | 1767.46 | 3320.76 | 5.2 | 436.3 | 1.0 | 176.7 | 332.1 | 951.3 | 0.5 | 45.9 | 0.1 | 18.6 | 34.9 | 22.1 |
| R92 | 0.015 | 0.18 | 0.06 | 22.90 | 0.00 | 7.62 | 9.90 | 0.0 | 2.3 | 0.0 | 0.8 | 1.0 | 4.0 | 0.2 | 56.6 | 0.0 | 18.8 | 24.5 | 17.9 |
| R93 | 0.015 | 0.16 | 0.06 | 24.21 | 0.24 | 4.58 | 62.20 | 0.0 | 2.4 | 0.0 | 0.5 | 6.2 | 9.1 | 0.1 | 26.5 | 0.3 | 5.0 | 68.1 | 16 |
| R103 | 0.015 | 0.15 | 0.11 | 51.91 | 0.00 | 0.00 | 0.00 | 0.0 | 5.2 | 0.0 | 0.0 | 0.0 | 5.2 | 0.2 | 99.8 | 0.0 | 0.0 | 0.0 | 15.8 |
| R105 | 0.015 | 0.15 | 0.04 | 29.14 | 0.00 | 0.00 | 0.00 | 0.0 | 2.9 | 0.0 | 0.0 | 0.0 | 2.9 | 0.1 | 99.9 | 0.0 | 0.0 | 0.0 | 15.9 |
| R107 | 0.015 | 0.16 | 0.49 | 32.10 | 0.00 | 12.12 | 0.00 | 0.0 | 3.2 | 0.0 | 1.2 | 0.0 | 4.5 | 1.1 | 71.8 | 0.0 | 27.1 | 0.0 | 9.5 |
| R109 | 0.015 | 0.15 | 0.76 | 66.10 | 0.21 | 174.44 | 26.28 | 0.1 | 6.6 | 0.0 | 17.4 | 2.6 | 26.8 | 0.3 | 24.7 | 0.1 | 65.1 | 9.8 | 17 |
| R116 | 0.015 | 0.15 | 1.08 | 49.81 | 0.63 | 24.08 | 507.56 | 0.1 | 5.0 | 0.1 | 2.4 | 50.8 | 58.3 | 0.2 | 8.5 | 0.1 | 4.1 | 87.1 | 21.6 |
| R124 | 0.015 | 0.16 | 3.08 | 110.91 | 0.36 | 30.94 | 193.96 | 0.3 | 11.1 | 0.0 | 3.1 | 19.4 | 33.9 | 0.9 | 32.7 | 0.1 | 9.1 | 57.2 | 14.9 |
| R133 | 0.015 | 0.15 | 0.06 | 32.90 | 0.35 | 2.94 | 23.50 | 0.0 | 3.3 | 0.0 | 0.3 | 2.3 | 6.0 | 0.1 | 55.1 | 0.6 | 4.9 | 39.3 | 16.2 |
| R134 | 0.015 | 0.15 | 0.05 | 36.92 | 0.30 | 2.55 | 37.95 | 0.0 | 3.7 | 0.0 | 0.3 | 3.8 | 7.8 | 0.1 | 47.5 | 0.4 | 3.3 | 48.8 | 4.7 |
| | | | | | | | | | | | | | AVG | 0.6 | 51.6 | 0.1 | 12.3 | 35.4 | 100.0 |

### Example 6: Selective Lithium Extraction From Synthetic Chile Brine

Synthetic Chile brine is a geothermal brine that contains a various amounts of dissolved metals. The composition of the Synthetic Chile brine used in the present study is shown in Table 4.

TABLE 5

Composition of metal ions in Synthetic Chile Brine at pH 7

| Synthetic Chile Brine | Composition | | | | Mass Ratios | | |
|---|---|---|---|---|---|---|---|
| | Li | Na | Mg | Ca | Na/Li | Mg/Li | Ca/Li |
| pH 7.0 (working pH about 8) | ppm | ppm | ppm | ppm | | | |
| | 500 | 20000 | 40000 | 40000 | 40 | 80 | 80 |

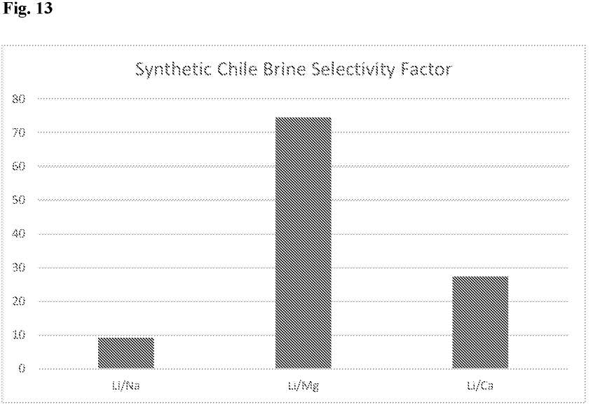

Extraction Selectivity: Selectivity is provided by comparing metal ion ratios in the eluted acidified aqueous solution (FIG. 13) to ion ratios in the feed solution (Table 5) for Synthetic Chile brine. FIG. 13 provides ratios for Li/Na, Li/Mg, and Li/Ca after treating the brine with an extractant disclosed herein using the protocol described above. In each case, lithium was selectively extracted using the liquid-liquid extraction method described herein even though the concentration of Na, Mg, and Ca in Salton Sea brine is substantially higher than the concentration of Li. Thus, the data shows that liquid-liquid extraction using compounds of the present disclosure is able to successfully enrich the aqueous acidified solution with lithium from Synthetic Chile brine.

### Example 7: Effect of Buffer on Lithium Extraction

TABLE 6

Comparison of brine composition, pH and density under buffered conditions

| Sample | Date | Ca | Fe | Mg | Mn | K | Na | Zn | U | pH | Density (g/mL) |
|---|---|---|---|---|---|---|---|---|---|---|---|
| Salton Sea Brine (neat) | May 24, 2018 | 323.00 | 1100 | 123 | 1180 | 18100 | 47400 | 468 | 246 | 0 | 1.25 |
| Degassed Brine* | Jul. 13, 2018 | 33700 | 316 | 91.5 | 1320 | 19700 | 64800 | 463 | 265 | 5.8 | 1.21 |
| 0.1 M Citric Acid Buffered Brine* | Jul. 13, 2018 | 30300 | 1.210 | 102 | 1280 | 20600 | 79100 | 481 | 281 | 5.8 | 1.24 |
| 0.1 M Acetic Acid Buffered Brine | Jul. 13, 2018 | 33800 | 804 | 105 | 1380 | 22700 | 68600 | 502 | 293 | 5.6 | 1.25 |
| 0.2 M Acetic Acid Buffered Brine pH 5.8* | Jul. 13, 2018 | 37900 | 728 | 11.3 | 1510 | 26500 | 68700 | 536 | 315 | 5.8 | 1.25 |
| 0.2 M Acetic Acid Suffered Brine pH 5.1* | Jul. 13, 2018 | 41200 | 825 | 117 | 1620 | 27800 | 80400 | 573 | 333 | 5.1 | 1.25 |

Buffered brine appears to have minimal impact on ion concentration and allows for the system to maintain its density. In some cases, pH adjustment resulted in precipitation (*). A number of small molecule extractants were tested under buffered conditions, including 1% compound 7 in 2-ethylhexanol (w/v). This compound was able to effectively extract lithium from a 0.1 M citric acid or a 0.2 M acetic acid buffered brine solution (Table 6). According to an analysis carried out as described above, 0.62 mg Li/g extractant and 0.38 mg Li/g of extractant were extracted in these two experiments, respectively. In both cases, 1 M HCl was used for the elution.

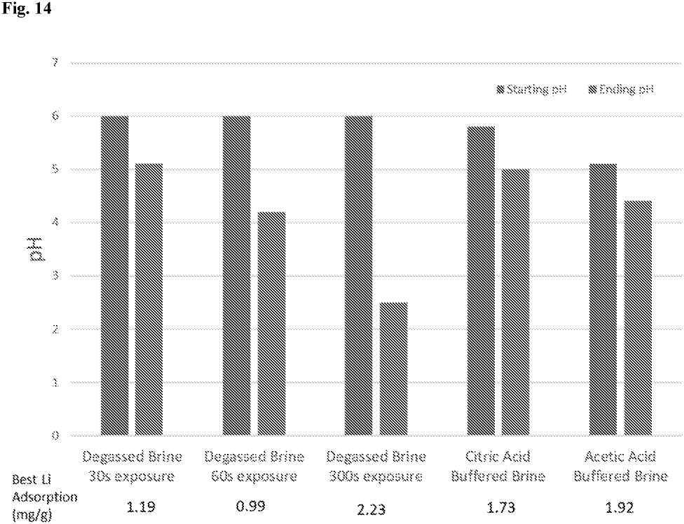

FIG. 14 shows how pH changes after extraction of brine. Buffered solutions are better able to resist drops in pH, however the current buffers are not able to maintain pH above 5. Without buffer, pH drops rapidly. However, there seems to be a delay between pH drop and the stripping effect seen in other samples. This is most likely related to the kinetics of stripping at the given pH.

Several extractants were tested under different brine conditions and each performed effectively (Table 7). In addition to buffering with either citric acid or acetic acid, degassing also appears to be a viable option for extracting lithium from brine solutions.

TABLE 7

Lithium Extraction from degassed and buffered brine solutions

| Sample | pH | Brine/Buffer | Elution | Adsorbed Li (mg/g) |
|---|---|---|---|---|
| R 280 | 2.5 | degassed | 1 M HCl | 2.23 |
| R 267 | 4.4 | acetic acid | 1 M HNO3 | 1.92 |
| R 279 | 2.6 | degassed | 1 M HCl | 1.86 |
| R 248 | 5.0 | citric acid | 1 M HCl | 1.73 |
| R 284 | 5.1 | degassed | 1 M HCl | 1.19 |
| R 283 | 4.9 | degassed | 1 M HCl | 1.16 |
| R 272 | 5.0 | citric acid | 1 M HNO3 | 1.13 |
| R 256 | 4.6 | acetic acid | 1 M H2SO4 | 1.13 |

### Embodiments of the Present Disclosure

1. A lithium-extracting polymer comprising at least one lithium chelating group, wherein the lithium capacity of the polymer is at least about 2 mg Li/g polymer (dry weight); the solubility of the polymer in diluent (e.g., 2-ethyl-1-hexanol) is at least about 100 g/L diluent and the polymer’s partition coefficient in a mixture of diluent:water is at least 10.

2. The polymer of embodiment 1, wherein the lithium capacity of the polymer is at least about 4 mg Li/g polymer.

3. The polymer of embodiment 1, wherein the lithium capacity of the polymer is at least about 10 mg Li/g polymer.

4. The polymer of any one of embodiments 1-3, wherein the polymer’s partition coefficient in a mixture of [diluent]:water is at least 100.

5. The polymer of any one of embodiments 1-4, wherein the polymer’s partition coefficient in a mixture of [diluent]:water is at least 1000.

6. The polymer of any one of embodiments 1-5, wherein the polymer’s molecular weight (MW) is from about 500 g/mol to about 50,000 g/mol

7. The polymer of any one of embodiments 1-5, wherein the polymer’s MW is from about 500 g/mol to about 15,000 g/mol.

8. The polymer of any one of embodiments 1-5, wherein the polymer’s MW is from about 500 g/mol to about 5,000 g/mol.

9. The polymer of any one of embodiments 1-8, wherein the use of the polymer in at least ten lithium ion liquid/liquid extraction elution cycles at a temperature of about 100° C. provides less than about 10% polymer degradation.

10. The polymer of any one of embodiments 1-8, wherein the use of the polymer in at least thirty lithium ion liquid/liquid extraction elution cycles using an extraction temperature of about 100° C., provides less than about 10% polymer degradation.

11. The polymer of embodiment 1, wherein the use of the polymer in at least one hundred lithium ion liquid/liquid extraction elution cycles using an extraction temperature of about 100° C. provides less than about 10% polymer degradation.

12. The polymer of any one of embodiments 1-8, wherein the use of the polymer in at least ten lithium ion liquid/liquid extraction elution cycles with a source phase having a pH of about 5 to 6 provides less than about 10% polymer degradation.

13. The polymer of any one of embodiments 1-8, wherein the use of the polymer in at least thirty lithium ion liquid/liquid extraction elution cycles with a source phase having a pH of about 5 to 6 provides less than about 10% polymer degradation.

14. The polymer of any one of embodiments 1-8, wherein the use of the polymer in at least one hundred lithium ion liquid/liquid extraction elution cycles with a source phase having a pH of about 5 to 6 provides less than about 10% polymer degradation.

15. The polymer of any one of embodiments 1-8, wherein the use of the polymer in at least ten lithium ion liquid/liquid extraction elution cycles with a source phase having a pH of at least about 10 provides less than about 10% polymer degradation.

16. The polymer of any one of embodiments 1-8, wherein the use of the polymer in at least thirty lithium ion liquid/liquid extraction elution cycles with a source phase having a pH of at least about 10 provides less than about 10% polymer degradation.

17. The polymer of any one of embodiments 1-8, wherein the use of the polymer in at least one hundred lithium ion liquid/liquid extraction elution cycles with a source phase having a pH of at least about 10 provides less than about 10% polymer degradation.

18. The polymer of any one of embodiments 1-17, wherein the flash point of the polymer is > 80° C.

19. The polymer of any one of embodiments 1-18, wherein the selectivity coefficient of the polymer for the target metal ion greater than about 5.

20. The polymer of any one of embodiments 1-19, wherein the lithium chelating group comprises one or more linear or macrocyclic polyether, polyamine, or polythioether ligand(s), including crown ethers, lariat ethers, multiarmed ethers, cryptands, calixarenes, and spherands.

21. The polymer of any one of embodiments 1-20, wherein the lithium chelating group comprises a 12-crown-4 polyether, a 12-crown-4 polyether polyamine, a 14-crown-4 polyether or a 14-crown-4 polyamine.

22. A polymer of Formula (III), prepared by a process comprising polymerizing a compound of Formula (I-C3) and a compound of Formula (II):

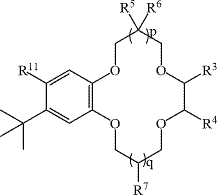

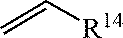

wherein:
R³ and R⁴ are each independently H, alkyl, alkene, optionally substituted aryl or optionally substituted cycloalkyl; or
R³ and R⁴ taken together with the carbon atoms to which they are attached form a cycloalkyl or aryl ring, each of which is optionally substituted;
R⁵ is H or alkyl;
R⁶ is —(CH₂)ᵣOH, -(CH₂)ᵣO-alkyl, —OH, —O—(CH₂)ₜC(O)OR⁸, —O—(CH₂)ₜS(O)₂OR⁸, —O—(CH₂)ₜS(O)₂N(R⁸)₂, —O—(CH₂)ₜP(O)₂(OR⁸)₂, —O—(CH₂)ₜC(O)N(R⁹)₂, each of which is optionally substituted;
R⁷ is H, —OH, -O-alkyl, -O-alkenyl, -O-alkynyl, or -O-cycloalkyl;
R⁸ is each independently H, alkyl, alkenyl, alkynyl, cycloalkyl, aryl, alkylene-cycloalkyl, or alkylene-aryl;
R⁹ is each independently H, alkyl, alkenyl, alkynyl, cycloalkyl, aryl, alkylene-cycloalkyl, alkylene-aryl, or SO₂R¹⁰;
R¹⁰ is alkyl, cycloalkyl, or haloalkyl;
R¹¹ is each independently H, alkyl, haloalkyl, alkene, alkyne, cycloalkyl, or aryl;
R¹³ is H, Cl, OH, alkyl, -O-alkyl, or aryl;
r is 1, 2, or 3;
t is independently 0, 1, or 2;
u is independently 1, 2, or 3;
with the proviso that either R⁷ is -O-alkenyl or R¹¹ is -alkenyl; and
R¹⁴ is optionally substituted aryl or optionally substituted heteroaryl.

23. The polymer of embodiment 22, wherein p and q are 0.

24. The polymer of embodiment 22, wherein p and q are 1.

25. The polymer of any one of embodiments 22-24, wherein R³ and R⁴ are H.

26. The polymer of any one of embodiments 22-24, wherein R³ and R⁴ taken together with the carbon atoms to which they are attached form an optionally substituted aryl ring.

27. The polymer of embodiment 26, wherein the optional substituent is selected from the group consisting of halogen, alkyl, haloalkyl, alkenyl, and cycloalkyl.

28. The polymer of embodiment 27, wherein the halogen is F or Cl; the alkyl is a C₁₋₆alkyl; the haloalkyl is CF₃, CHF₂, CH₂F, or CH₂Cl; the alkenyl is a C₂₋₄alkenyl; and the cycloalkyl is a C₃₋₆cycloalkyl.

29. The polymer of embodiment 27 or 28, wherein the alkyl is t-butyl.

30. The polymer of embodiment 22, wherein p and q are 0, and R³ and R⁴ are H.

31. The polymer of any one of embodiments 22-30, wherein R¹¹ is alkenyl.

32. The polymer of embodiment 31, wherein the alkenyl is a C₂₋₁₂alkenyl.

33. The polymer of embodiment 31 or 32, wherein the alkenyl is vinyl.

34. The polymer of any one of embodiments 22-33, wherein R⁷ is H, alkyl, —OH or -O-alkyl.

35. The polymer of embodiment 34, wherein the alkyl is hexyl.

36. The polymer of embodiment 22-30, wherein R⁷ is -O-alkenyl or -O-alkylene-SiR¹³.

37. The polymer of any one of embodiments 22-30, wherein R⁷ is -O-alkenyl, and the-O-alkenyl is —OCH₂CH═CH.

38. The polymer of embodiment 36 or 37, wherein R¹¹ is H.

39. The polymer of any one of embodiments 22-38, wherein R⁵ is H or hexyl.

40. The polymer of any one of embodiments 22-39, wherein R⁶ is selected from the group consisting of—OS(O)₂OH, —O(CH₂)ₜP(O)(OR⁸)(OH), —O(CH₂)ₜC(O)OH, —O(CH₂)ₜC(O)NH(SO₂CF₃) and optionally substituted —OPh.

41. The polymer of any one of embodiments 22-40, wherein t is 0 or 1.

42. The polymer of embodiment 40, wherein —OPh is optionally substituted with —C(O)N(H)S(O)₂R¹², wherein R¹² is selected from the group consisting of alkyl, haloalkyl, or cycloalkyl.

43. The polymer of embodiment 42, wherein R¹² is haloalkyl, and the haloalkyl is CF₃.

44. The polymer of embodiment 40, wherein the optionally substituted phenyl is

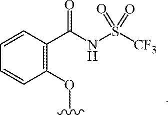

45. The polymer of any one of embodiments 22-44, wherein R⁸ is H, ethyl or phenyl.

46. The polymer of any one of embodiments 22-45, wherein R⁹ is SO₂R¹⁰, and R¹⁰ is C₁₋₅alkyl or haloalkyl selected from the group consisting of CF₃, CHF₂, and CH₂F.

47. The polymer of any one of embodiments 22-45, wherein R⁹ is SO₂R¹⁰, and R¹⁰ is CF₃.

48. The polymer of any one of embodiments 22-47, wherein each R¹¹ is independently H, alkyl, haloalkyl, or cycloalkyl.

49. The polymer of any one of embodiments 22-48, wherein R¹⁴ is phenyl.

50. The polymer of any one of embodiments 1-49, wherein the lithium chelating is selected from the group consisting of 4-hydroxyl-bis(4′-t-butyl)dibenzo-14-crown-4 ether, 4,11-dihydroxyl-bis(4′-t-butyl)dibenzo-14-crown-4 ether, (4′-t-butyl)benzo-12-crown-4 ether, (4′-t-butyl)cyclohexyl-12-crown-4 ether, bis(4′-t-butyl)dibenzo-14-crown-4 ether, bis(4′-t-butyl)dicyclohexyl-14-crown-4 ether, 4-alkylhydroxyl-bis(4′-t-butyl)dibenzo-14-crown-4 ether, 4,11-dialkylhydroxyl-bis(4′-t-butyl)dibenzo-14-crown-4 ether, sym(4′-t-butyl)dibenzo-14-crown-4-oxyacetic acid ether, sym(4′-t-butyl)dibenzo-14-crown-4-oxysulfuric acid ether, sym(4′-t-butyl)dibenzo-14-crown-4-oxyphenylphosphonic acid ether, or sym(4′-t-butyl)dibenzo-14-crown-4-oxy-N-((trifluoromethyl)sulfonyl)acetamide ether.

51. The polymer of any one of embodiments 1-50, wherein one or more of the following groups is attached at one or more points along the polyether or polyamine linear and/or macrocyclic chains: phenyl, aromatic, linear or branched alkyl, cyclohexyl, ether, polyether, poly(ethylene oxide), polypropylene oxide), amine, polyamine, phosphate, phosphite, carboxylic acid derivative, phosphonic acid derivative, sulfonic acid derivative, amino acid derivative, trifluoromethylsulfonyl carbamoyl, or other proton-ionizable group.

52. The polymer of embodiment 1, wherein the polymer has the structural formula:

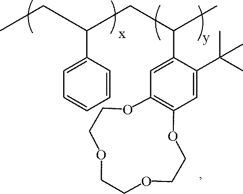

wherein x is an integer between 0 and 10 and y is an integer between 1 and 10.

53. The polymer of any one of embodiments 1-52, wherein the polymer is prepared by the polymerization of one or more lithium chelating monomers functionalized with a polymerizable group.

54. The polymer of any one of embodiments 1-53, wherein the polymerizable group is selected from the group consisting of a vinyl, chlorosilane, or silanol group.

55. The polymer of any one of embodiments 1-54, wherein the polymerizable group is a vinyl group attached to an aromatic or phenyl group.

56. The polymer of any one of embodiments 1-55, wherein the polymerizable group is polymerized via thermal, photo, hydrolysis and condensation, or other catalytic and non-catalytic mediated initiation.

57. The polymer of any one of embodiments 1-56, wherein the one or more lithium chelating monomers are polymerized with one or more non-ligand monomers.

58. The polymer of any one of embodiments 1-57, wherein the polymer is prepared by the polymerization of one or more lithium chelating monomers selected from the group consisting of:

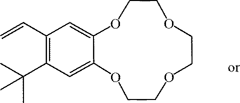

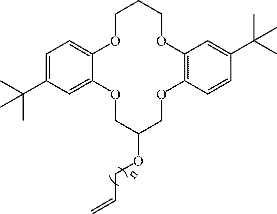

wherein X is selected from H, Cl, OH, alkyl, alkoxy, or aromatic, and n is an integer from 1 to 12 or mixtures thereof.

59. A plurality of macroreticular polymer beads comprising a copolymer having a plurality of complexing cavities which selectively bind lithium ion, wherein the copolymer comprises one or more lithium chelating monomers.

60. The macroreticular beads of embodiment 59, further comprising a non-ligand monomer, or a crosslinking monomer, or a mixture thereof.

61. The macroreticular beads of embodiment 60, wherein the weight ratio of lithium chelating monomers to non-ligand monomer and crosslinking monomer is at least about 5:1

62. The macroreticular beads of any one of embodiments 59-61, wherein the lithium chelating monomer is selected from the group consisting of sym(4′-t-butyl)dibenzo-14-crown-4-oxyallyl ether, (4′-t-butyl-3′-vinyl)benzo-12-crown-4 ether, sym(4′-t-butyl)dibenzo-14-crown-4-oxyalkylallyl ether, sym(4′-t-butyl)dibenzo-14-crown-4-alkylallyl ether, sym(4′-t-butyl)dibenzo-14-crown-4-(oxydialkoxy silane) ether, and sym(4′-t-butyl)dibenzo-14-crown-4--(oxyalkyldialkoxy silane) ether.

63. The macroreticular beads of any one of embodiments 59-62, wherein the copolymer comprises about 0.1 to about 10 mole percent of the crosslinking monomer.

64. The macroreticular beads of any one of embodiments 59-63, wherein the macroreticular beads have a surface area of about 0.1-500 m²/g.

65. The macroreticular beads of any one of embodiments 59-64, wherein the macroreticular beads have an average particle size of from about 250 µm to about 1.5 mm.

66. The macroreticular beads of any one of embodiments 59-65, wherein the use of the beads in at least ten lithium ion extraction elution cycles at a temperature of about 100° C. provides less than about 10% polymer degradation.

67. The macroreticular beads of any one of embodiments 59-66, wherein the use of the beads in at least thirty lithium ion extraction elution cycles at a temperature of about 100° C. provides less than about 10% polymer degradation.

68. The macroreticular beads of any one of embodiments 59-67, wherein the use of the beads in at least one hundred lithium ion extraction elution cycles using an extraction temperature of about 100° C. provides less than about 10% polymer degradation.

69. The macroreticular beads of any one of embodiments 59-68, wherein the use of the beads in at least ten lithium ion extraction elution cycles with a source phase having a pH of about 5 to 6 provides less than about 10% polymer degradation.

70. The macroreticular beads of any one of embodiments 59-69, wherein the use of the beads in at least thirty lithium ion extraction elution cycles with a source phase having a pH of about 5 to 6 provides less than about 10% polymer degradation.

71. The macroreticular beads of any one of embodiments 59-70, wherein the use of the beads in at least one hundred lithium ion extraction elution cycles with a source phase having a pH of about 5 to 6 provides less than about 10% polymer degradation.

72. The macroreticular beads of any one of embodiments 59-71, wherein the flash point of the polymer is > 80° C.

73. The macroreticular beads of any one of embodiments 59-72, wherein the selectivity coefficient of the beads for the target metal ion greater than about 5.

74. A sorbent comprising a solid support and a lithium chelating group.

75. The sorbent of embodiment 74, wherein the lithium chelating group is coated on the solid support.

76. The sorbent of embodiment 74, wherein the lithium chelating group is chemically linked to the solid support.

77. The sorbent of any one of embodiments 74-76, wherein the solid support is selected from the group consisting of silica, alumina, titania, manganese oxide, glass, zeolite, lithium ion sieve, molecular sieve, or other metal oxide.

78. The sorbent of any one of embodiments 74-77, wherein the sorbent has a surface area of about 0.1-500 m²/g.

79. The sorbent of any one of embodiments 74-78, wherein the sorbent has an average particle size of from about 250 µm to about 1.5 mm.

80. The sorbent of any one of embodiments 74-79, wherein the use of the sorbent in at least ten lithium ion extraction elution cycles at a temperature of about 100° C. provides less than about 10% polymer degradation.

81. The sorbent of any one of embodiments 74-80, wherein the use of the sorbent in at least thirty lithium ion extraction elution cycles at a temperature of about 100° C. provides less than about 10% polymer degradation.

82. The sorbent of any one of embodiments 74-81, wherein the use of the sorbent in at least one hundred lithium ion extraction elution cycles using an extraction temperature of about 100° C. provides less than about 10% polymer degradation.

83. The sorbent of any one of embodiments 74-82, wherein the use of the sorbent in at least ten lithium ion extraction elution cycles with a source phase having a pH of about 5 to 6 provides less than about 10% polymer degradation.

84. The sorbent of any one of embodiments 74-83, wherein the use of the sorbent in at least thirty lithium ion extraction elution cycles with a source phase having a pH of about 5 to 6 provides less than about 10% polymer degradation.

85. The sorbent of any one of embodiments 74-84, wherein the use of the sorbent in at least one hundred lithium ion extraction elution cycles with a source phase having a pH of about 5 to 6 provides less than about 10% polymer degradation.

86. The sorbent of any one of embodiments 74-85, wherein the flash point of the polymer is > 80° C.

87. The sorbent of any one of embodiments 74-86, wherein the selectivity coefficient of the sorbent for the target metal ion greater than about 5.

88. A method of extracting lithium, comprising:
(a) mixing a lithium-containing aqueous phase with an organic phase comprising a suitable organic solvent and one or more polymers of embodiments 1-29, macroreticular beads of any one of embodiments 30-44 or sorbent of any one of embodiments 45-58, or a mixture thereof;
(b) separating the organic phase and the aqueous phase; and
(c) treating the organic phase with acidic solution to yield a lithium salt solution.

89. The method of embodiment 88, wherein the suitable solvent is selected from the group consisting of alcohols, aldehydes, alkanes, amines, amides, aromatics, carboxylic acids, ethers, ketones, phosphates, or siloxanes or a mixture thereof.

90. The method of embodiment 88 or 89, wherein the aqueous phase is selected from the group consisting of natural brine, a dissolved salt flat, seawater, concentrated seawater, desalination effluent, a concentrated brine, a processed brine, a geothermal brine, liquid from an ion exchange process, liquid from a solvent extraction process, a synthetic brine, leachate from ores, leachate from minerals, leachate from clays, leachate from recycled products, leachate from recycled materials, or combination thereof.

91. The method of embodiments 88-90, wherein the aqueous phase is a geothermal brine.

92. The method of any one of embodiments 88-91, wherein the acid solution comprises one or more of hydrochloric acid, sulfuric acid, phosphoric acid, hydrobromic acid, chloric acid, perchloric acid, nitric acid, formic acid, acetic acid, carbonic acid, or a combination thereof.

93. A method of preparing a macroreticular bead, comprising polymerizing:
(a) a lithium chelating monomer;
(b) an optional non-ligand monomer; and
(c) a crosslinking monomer.

94. The method of embodiment 93, wherein the polymerization is carried out by reverse phase suspension polymerization.

95. A method of preparing a sorbent, comprising:
(a) coating a solid support with lithium chelating group or
(b) chemically linking lithium chelating group to a solid support.

96. A method of selectively sequestering one or more target metal ions from a solution of the one or more metal ion ions admixed with other ions, comprising contacting one or more macroreticular polymer beads of any one of embodiments 59-73 or sorbents of any one of embodiments 74-87 with a stripping solution, whereby the complexed ions are removed from the macroreticular polymer beads, then contacting the stripped beads with the solution, thereby selectively sequestering the target ion in the macroreticular polymer beads.

1. A compound of Formula (I): 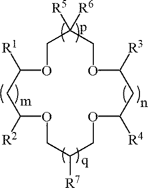
wherein
R¹, R², R³, and R⁴ are each independently H, alkyl, alkenyl, alkynyl, cycloalkyl, aryl, or heteroaryl, each of which are optionally substituted; or
R¹ and R² and/or R³ and R⁴ taken together with the carbon atoms to which they are attached form a cycloalkyl or aryl ring, each of which is optionally substituted;
R⁵ when present is H, alkyl, alkenyl, alkynyl, or cycloalkyl;
R⁶ when present is —(CH₂)ᵣOH, -(CH₂)ᵣO-alkyl, —OH, -O-alkyl, -O-alkenyl, -O-alkynyl, -O-cycloalkyl; -O-aryl, —O—(CH₂)ₜC(O)OR⁸, —O—(CH₂)ₜS(O)₂OR⁸, —O—(CH₂)ₜS(O)₂N(R⁸)₂, —O—(CH₂)ₜP(O)(OR⁸)₂, —O—(CH₂)ₜC(O)N(R⁹)₂, each of which is optionally substituted;
R⁷ is H, —OH, -O-alkyl, -O-alkenyl, -O-alkynyl, -O-cycloalkyl, —O—(CH₂)ₜC(O)OR⁸, —O—(CH₂)ₜS(O)₂OR⁸, —O—(CH₂)ₜS(O)₂N(R⁸)₂, —O—(CH₂)ₜP(O)(OR⁸)₂, or —O—(CH₂)ₜC(O)N(R⁹)₂; R⁸ is each independently H, alkyl, haloalkyl, alkenyl, alkynyl, cycloalkyl, aryl, alkylene-cycloalkyl, or alkylene-aryl;
R⁹ is each independently H, alkyl, alkenyl, alkynyl, cycloalkyl, aryl, alkylene-cycloalkyl, alkylene-aryl, or SO₂R¹⁰;
R¹⁰ is alkyl, cycloalkyl, or haloalkyl;
m, n, p, and q are each independently 0 or 1;
r is 1, 2, or 3; and
t is independently 0, 1, or 2;
with the proviso that when p is 0, at least two of R¹, R², R³, and R⁴ are not H.

2. The compound of claim 1, wherein m and n are each 0.

3. The compound of claim 1, wherein p and q are each 1.

4. The compound of claim 1, wherein p and q are each 0.

5-6. (canceled)

7. The compound of claim 1, wherein R¹ and R² taken together with the carbon atoms to which they are attached form a cycloalkyl or aryl ring, each of which is optionally substituted.

8. (canceled)

9. The compound of claim 1, wherein R³ and R⁴ taken together with the carbon atoms to which they are attached form a cycloalkyl or aryl ring, each of which is optionally substituted.

10. (canceled)

11. The compound of claim 7, wherein the cycloalkyl ring is an optionally substituted cyclohexyl.

12. The compound of claim 7, wherein the aryl ring is an optionally substituted phenyl.

13. The compound of claim 7, wherein the optional substituent is selected from the group consisting of halogen, alkyl, haloalkyl, alkenyl, and cycloalkyl.

14. The compound of claim 13, wherein the halogen is F or Cl; the alkyl is a C₁₋₆alkyl; the haloalkyl is CF₃, CHF₂, CH₂F, or CH₂Cl; the alkenyl is a C₂₋₄alkenyl; and the cycloalkyl is a C₃₋₆cycloalkyl.

15. The compound of claim 14, wherein the C₁₋₆alkyl is methyl, ethyl, propyl, i-propyl, butyl, isobutyl, t-butyl, or t-amyl.

16. The compound of claim 14 , wherein the C₁₋₆alkyl is t-butyl.

17-21. (canceled)

22. The compound of claim 1, wherein R⁶ is selected from the group consisting of —OS(O)₂OH, —O(CH₂)ₜP(O)(OR⁸)(OH), —O(CH₂)ₜC(O)OH, —O(CH₂)ₜC(O)NH(R⁹) and optionally substituted —OPh.

23-26. (canceled)

27. The compound of claim 1, wherein R⁷ is H, alkyl, —OH, -O-alkyl, —O—(CH₂)ₜC(O)OR⁸, —O—(CH₂)ₜS(O)₂OR⁸, or —O—(CH₂)ₜP(O)(OR⁸)₂.

28-32. (canceled)

33. The compound of claim 1, wherein the compound of Formula (I) is a compound of Formula (I-B1) or Formula (I-B2): wherein 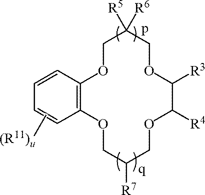 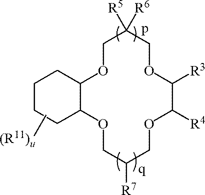
R³ and R⁴ are each independently H, alkyl, alkene, optionally substituted aryl or optionally substituted cycloalkyl; or
R³ and R⁴ taken together with the carbon atoms to which they are attached form a cycloalkyl or aryl ring, each of which is optionally substituted;
R⁵ is H, alkyl, alkenyl, alkynyl, or cycloalkyl;
R⁶ is —(CH₂)ᵣOH, -(CH₂)ᵣO-alkyl, —OH, -O-alkyl, -O-alkenyl, -O-alkynyl, -O-cycloalkyl; -O-aryl, —O—(CH₂)ₜC(O)OR⁸, —O—(CH₂)ₜS(O)₂OR⁸, —O—(CH₂)ₜS(O)₂N(R⁸)₂, —O—(CH₂)ₜP(O)₂(OR⁸)₂, —O—(CH₂)ₜC(O)N(R⁹)₂, each of which is optionally substituted;
R⁷ is H, —OH, -O-alkyl, -O-alkenyl, -O-alkynyl, -O-cycloalkyl, —O—(CH₂)ₜC(O)OR⁸, —O—(CH₂)ₜS(O)₂OR⁸, —O—(CH₂)ₜS(O)₂N(R⁸)₂, —O—(CH₂)ₜP(O)(OR⁸)₂, or —O—(CH₂)ₜC(O)N(R⁹)₂;
R⁸ is each independently H, alkyl, alkenyl, alkynyl, cycloalkyl, aryl, alkylene-cycloalkyl, or alkylene-aryl;
R⁹ is each independently H, alkyl, alkenyl, alkynyl, cycloalkyl, aryl, alkylene-cycloalkyl, alkylene-aryl, or SO₂R¹⁰;
R¹⁰ is alkyl, cycloalkyl, or haloalkyl;
R¹¹ is each independently H, alkyl, haloalkyl, alkene, alkyne, cycloalkyl, or aryl;
p and q are each independently 0 or 1;
r is 1, 2, or 3;
t is independently 0, 1, or 2; and
u is 0, 1, 2, or 3.

34-58. (canceled)

59. The compound of claim 1, wherein the compound of Formula (I) is a compound of Formula (I-C1) or Formula (I-C2): 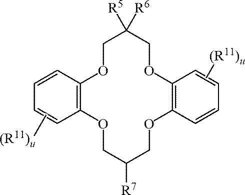 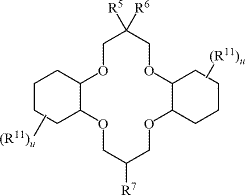
wherein
R⁵ is H, alkyl, alkenyl, alkynyl, or cycloalkyl;
R⁶ is H, —(CH₂)ᵣOH, -(CH₂)ᵣO-alkyl, —OH, -O-alkyl, -O-alkenyl, -O-alkynyl, -O-cycloalkyl; -O-aryl, —O—(CH₂)ₜC(O)OR⁸, —O—(CH₂)ₜS(O)₂OR⁸, —O—(CH₂)ₜS(O)₂N(R⁸)₂, —O—(CH₂)ₜP(O)₂(OR⁸)₂, —O—(CH₂)ₜC(O)N(R⁹)₂, each of which is optionally substituted;
R⁷ is H, —OH, -O-alkyl, -O-alkenyl, -O-alkynyl, or -O-cycloalkyl;
R⁸ is each independently H, alkyl, alkenyl, alkynyl, cycloalkyl, aryl, alkylene-cycloalkyl, or alkylene-aryl;
R⁹ is each independently H, alkyl, alkenyl, alkynyl, cycloalkyl, aryl, alkylene-cycloalkyl, alkylene-aryl, or SO₂R¹⁰;
R¹⁰ is alkyl, cycloalkyl, or haloalkyl;
R¹¹ is each independently H, alkyl, haloalkyl, alkene, alkyne, cycloalkyl, or aryl;
r is 1, 2, or 3;
t is independently 0, 1, or 2; and
u is independently 0, 1, 2, or 3.

60-74. (canceled)

75. The compound of claim 1, wherein the compound of Formula (I) is selected from the group consisting of: 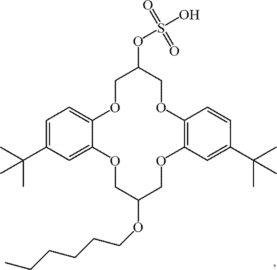 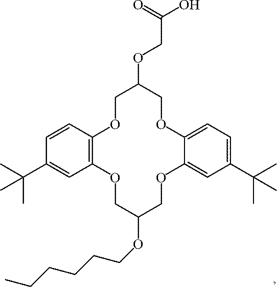 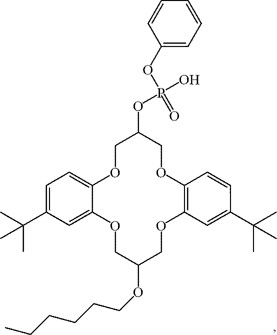 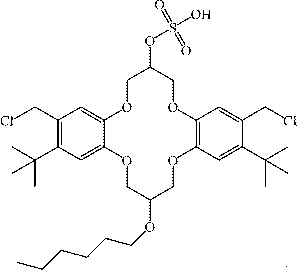 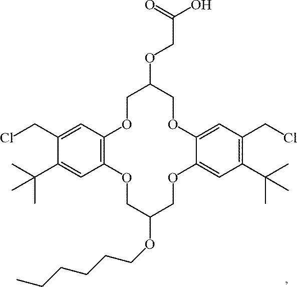 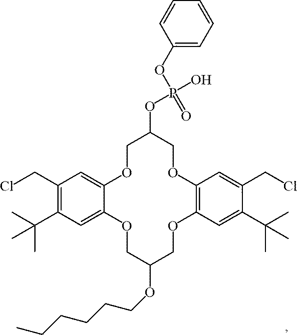 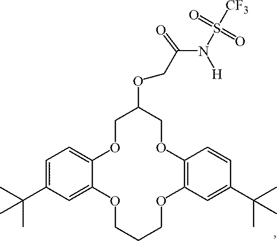 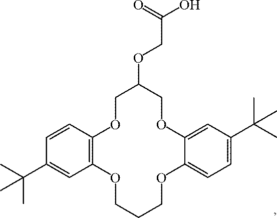 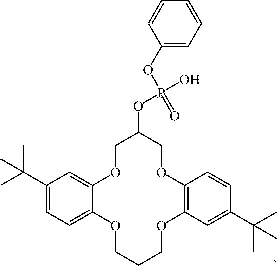 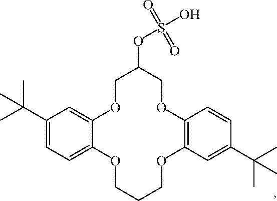 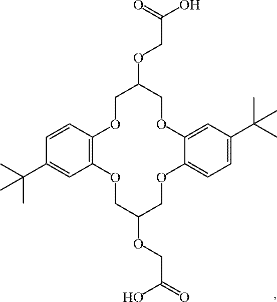 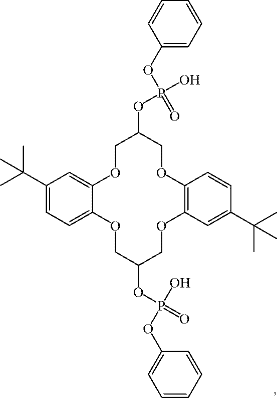 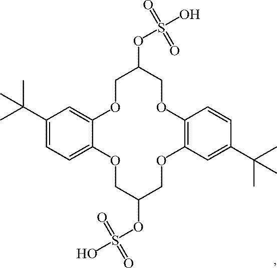 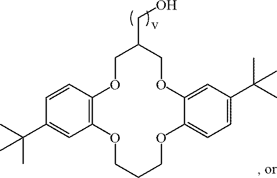 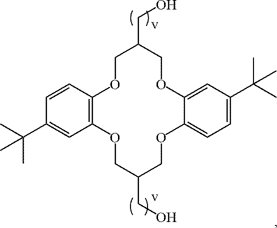
wherein each v is independently 0, 1, 2, or 3.

76. A method of extracting lithium, comprising:
(a) mixing a lithium-containing aqueous phase with an organic phase comprising a suitable organic solvent and one or more compounds of claim 1,
(b) separating the organic phase and the aqueous phase; and
(c) treating the organic phase with aqueous acidic solution to yield a aqueous lithium salt solution.

77-79. (canceled)

80. The method of claim 76 , wherein the aqueous phase is selected from the group consisting of natural brine, a dissolved salt flat, seawater, concentrated seawater, desalination effluent, a concentrated brine, a processed brine, a geothermal brine, liquid from an ion exchange process, liquid from a solvent extraction process, a synthetic brine, leachate from ores, leachate from minerals, leachate from clays, leachate from recycled products, leachate from recycled materials, or combination thereof.

81. The method of claim 76, wherein the aqueous phase is a geothermal brine.
